SEQUENCE LISTING (35 sequences)

SEQ ID NO: 1 - DNA, 31 bp, Artificial
  Synthetic
acgttggatggttatggtccttaggaaaatg

SEQ ID NO: 2 - DNA, 31 bp, Artificial
  Synthetic
acgttggatgggcaacgtctatagatttacc

SEQ ID NO: 3 - DNA, 30 bp, Artificial
  Synthetic
acgttggatggaccctggatgtttccttac

SEQ ID NO: 4 - DNA, 30 bp, Artificial
  Synthetic
acgttggatggccaatataggatagggcac

SEQ ID NO: 5 - DNA, 30 bp, Artificial
  Synthetic
acgttggatgttaacgcagatcgagttggg

SEQ ID NO: 6 - DNA, 30 bp, Artificial
  Synthetic
acgttggatgtttctgcactaggtctgcac

SEQ ID NO: 7 - DNA, 30 bp, Artificial
  Synthetic
acgttggatggccaattatcttacagggtc

SEQ ID NO: 8 - DNA, 30 bp, Artificial
  Synthetic
acgttggatgttgggaaagccttcagtcct

SEQ ID NO: 9 - DNA, 30 bp, Artificial
  Synthetic
acgttggatgtccctcctagagaagatctg

SEQ ID NO: 10 - DNA, 30 bp, Artificial
  Synthetic
acgttggatgcctggatccccaggaaaatg

SEQ ID NO: 11 - DNA, 29 bp, Artificial
  Synthetic
acgttggatgggagtctaactcatgcttc

SEQ ID NO: 12 - DNA, 30 bp, Artificial
  Synthetic
acgttggatgtgattccatcaatgcatggg

SEQ ID NO: 13 - DNA, 30 bp, Artificial
  Synthetic
acgttggatggagccatctcctgatgcttg

SEQ ID NO: 14 - DNA, 30 bp, Artificial
  Synthetic
acgttggatgcacacagacactcacagacg

SEQ ID NO: 15 - DNA, 25 bp, Artificial
  Synthetic
tttggaaaatggatataatcaaaat

SEQ ID NO: 16 - DNA, 26 bp, Artificial
  Synthetic
tttggaaaatggatataatcaaaatc

SEQ ID NO: 17 - DNA, 17 bp, Artificial
  Synthetic
taccagctccagggtgt

SEQ ID NO: 18 - DNA, 18 bp, Artificial
  Synthetic
taccagctccagggtgtc

SEQ ID NO: 19 - DNA, 21 bp, Artificial
  Synthetic
ggagcgagttgggaacgcacg

SEQ ID NO: 20 - DNA, 22 bp, Artificial
  Synthetic
ggagcgagttgggaacgcacgc

SEQ ID NO: 21 - DNA, 18 bp, Artificial
  Synthetic
tacagggtctttctccac

SEQ ID NO: 22 - DNA, 19 bp, Artificial
  Synthetic
tacagggtctttctccacc

SEQ ID NO: 23 - DNA, 17 bp, Artificial
  Synthetic
gcctaacagaggcttgg

SEQ ID NO: 24 - DNA, 18 bp, Artificial
  Synthetic
gcctaacagaggcttggc

SEQ ID NO: 25 - DNA, 20 bp, Artificial
  Synthetic
aacaactcacccctggtttc

SEQ ID NO: 26 - DNA, 21 bp, Artificial
  Synthetic
aacaactcacccctggtttca

SEQ ID NO: 27 - DNA, 18 bp, Artificial
  Synthetic
atgatgcttggtgttaga

SEQ ID NO: 28 - DNA, 19 bp, Artificial
  Synthetic
atgatgcttggtgttagac

SEQ ID NO: 29 - DNA, 26 bp, Artificial
  Synthetic
tttggaaaatggatataatcaaaatt

SEQ ID NO: 30 - DNA, 18 bp, Artificial
  Synthetic
taccagctccagggtgtt

SEQ ID NO: 31 - DNA, 22 bp, Artificial
  Synthetic
ggagcgagttgggaacgcacgg

SEQ ID NO: 32 - DNA, 19 bp, Artificial
  Synthetic
tacagggtctttctccact

SEQ ID NO: 33 - DNA, 18 bp, Artificial
  Synthetic
gcctaacagaggcttgga

SEQ ID NO: 34 - DNA, 21 bp, Artificial
  Synthetic
aacaactcacccctggtttcg

SEQ ID NO: 35 - DNA, 19 bp, Artificial
  Synthetic
atgatgcttggtgttagat

(19) United States
(12) Patent Application Publication
(10) Pub. No.: US 2010/0009368 A1
(43) Pub. Date: Jan. 14, 2010

(54) METHODS AND COMPOSITIONS FOR THE ASSESSMENT OF CARDIOVASCULAR FUNCTION AND DISORDERS

(75) Inventor: Robert Peter Young, Auckland (NZ)

Correspondence Address: DAVIS WRIGHT TREMAINE LLP - San Francisco, 505 MONTGOMERY STREET, SUITE 800, SAN FRANCISCO, CA, 94111, US

(73) Assignee: Synergenz Bioscience Limited, Tortola (VG)

(21) Appl. No.: 12/488,461

(22) Filed: Jun. 19, 2009

Related U.S. Application Data
(63) Continuation-in-part of application No. PCT/NZ2007/000368, filed on Dec. 19, 2007.

(30) Foreign Application Priority Data
Dec. 19, 2006 (NZ) 552236

(51) Int. Cl. C12Q 1/68; C07H 21/04; C40B 40/08; C40B 40/10

(52) U.S. Cl. 435/6; 536/24.3; 536/24.33; 506/17; 506/18

(57) ABSTRACT

The present invention provides methods for the assessment of risk of developing acute coronary syndrome (ACS), arterial inflammation, or ACS-associated impaired vascular function, in smokers and non-smokers using analysis of genetic polymorphisms. The present invention also relates to the use of genetic polymorphisms in assessing a subject's risk of developing ACS, arterial inflammation, or ACS-associated impaired vascular function. Nucleotide probes and primers, kits, and microarrays suitable for such assessment are also provided.

### CROSS-REFERENCE TO RELATED APPLICATIONS

The present application is a continuation-in-part of International Application No. PCT/NZ2007/000368, filed Dec. 19, 2007, designating the United States of America and published in English on Jun. 26, 2008, which in turn claims priority to New Zealand Patent Application No. 552236, filed Dec. 19, 2006, each of the foregoing which is incorporated herein by reference in its entirety.

### FIELD OF THE INVENTION

The present invention is concerned with methods for assessment of vascular function and/or disorders, and in particular for diagnosing predisposition to and/or severity of coronary artery disease and particularly acute coronary syndrome (ACS) using analysis of genetic polymorphisms and altered gene expression. The present invention is also concerned with methods for diagnosing predisposition to and/or severity of ACS-associated impaired vascular function.

### BACKGROUND OF THE INVENTION

Coronary artery disease (CAD), also known as coronary heart disease or arteriosclerotic heart disease, is the leading cause of death in the United States. According to the American Heart Association, about every 29 seconds someone in the US suffers from a CAD-related event, and about every minute someone dies from such an event. The lifetime risk of having coronary heart disease after age 40 is 49% for men and 32% for women. As women age, the risk increases almost to that of men. Furthermore, the total annual cost of CAD in the United States is approximately US$130 billion.

The cardiovascular disorders that underlie CAD can be divided into two groups, as indeed can the sufferers of such disorders. This is thought to reflect different etiology of the disorders. The disorders of the first group, herein referred to as “Stable CAD”, are degenerate in nature and include the late onset and exertional anginas. Stable CAD typically afflicts older persons, and is associated with age (65 and greater), high blood pressure, diabetes, high cholesterol levels (specifically, high LDL cholesterol and low HDL cholesterol), lack of physical activity or exercise, and obesity.

The disorders of the second group, herein referred to as acute coronary syndrome (ACS), are believed to be associated with inflammation, plaque instability, and/or smoking. ACS includes myocardial infarction and unstable angina. See, for example, Mulvihill N T and Foley J B “Inflammation in acute coronary syndromes” Heart 2002;87:201-204; Libby P “Current Concepts of the Pathogenesis of the Acute Coronary Syndromes” Circulation 2001; 104:365-372; Libby P and Theroux P “Pathophysiology of Coronary Artery Disease” Circulation 2005;111:3481-3488. The Applicants believe, without wishing to be bound by any theory, that, more so than in Stable CAD, genetic risk factors are significant in susceptibility to and/or severity of ACS.

Moreover, the Applicants believe, again without wishing to be bound by any theory, that the biomarkers associated with Stable CAD are unlikely to be associated with, or predictive of, risk of ACS, and vice versa.

It would be desirable and advantageous to have biomarkers which could be used to assess a subject's risk of developing acute coronary syndrome (ACS), risk of developing ACS-associated impaired vascular function, arterial inflammation, or other symptoms associated with ACS, particularly if the subject is a smoker.

It is primarily to such biomarkers and their use in methods to assess risk of developing such disorders that the present invention is directed.

### BRIEF DESCRIPTION OF THE INVENTION

The present invention is primarily directed to determining the association between genotypes and the subject's risk of developing acute coronary syndrome (ACS). As used herein, ACS includes but is not limited to myocardial infarction, unstable angina, and related acute coronary syndromes.

Thus, according to one aspect there is provided a method of determining a subject's risk of developing ACS, arterial inflammation, or ACS-associated impaired vascular function, the method comprising analyzing a sample from said subject for the presence or absence of one or more polymorphisms selected from the group consisting of:
Y402H C/T (rs1061170) in the gene encoding Complement Factor H (CFH);
Asp92Asn A/G (rs11666735) in the gene encoding Myeloid IgA Fc receptor (FCAR);
A/G (rs4804611) in the gene encoding Zinc finger protein 627 (ZNF627);
Asn159Asn A/G (rs6747096) in the gene encoding Serpin 2; or
C3279T A/G (rs7291467) in the gene encoding Galectin-2 (LGALS2);

wherein the presence or absence of one or more of said polymorphisms is indicative of the subject's risk of developing ACS, arterial inflammation, or ACS-associated impaired vascular function.

The one or more polymorphisms can be detected directly or by detection of one or more polymorphisms which are in linkage disequilibrium with said one or more polymorphisms.

Linkage disequilibrium (LD) is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204.) The method can additionally comprise analyzing a sample from said subject for the presence of one or more further polymorphisms selected from the group consisting of:
A387P C/G (rs1866389) in the gene encoding Thrombospondin 4; or
Asp51Ala A/C (rs6743376) in the gene encoding Interleukin 1 family, member 10 (ILIF10).

Again, detection of the one or more further polymorphisms may be carried out directly or by detection of polymorphisms in linkage disequilibrium with the one or more further polymorphisms.

The presence of one or more polymorphisms selected from the group consisting of:
the Asp92Asn A/G AA or AG genotype in the gene encoding Myeloid IgA Fc receptor (FCAR);
the A387P C/G GG genotype in the gene encoding Thrombospondin 4;
the A/G (rs4804611) AA genotype in the gene encoding Zinc finger protein 627 (ZNF627);
the Asn159Asn A/G AA genotype in the gene encoding Serpin 2; or
the C3279T A/G GG genotype in the gene encoding Galectin-2 (LGALS2) may be indicative of a decreased risk of developing ACS, arterial inflammation, or ACS-associated impaired vascular function.

The presence of one or more polymorphisms selected from the group consisting of:
the Y402H C/T TT genotype in the gene encoding Complement Factor H;
the Asp92Asn A/G GG genotype in the gene encoding Myeloid IgA Fc receptor (FCAR);
the A/G (rs4804611) GA or GG genotype in the gene encoding Zinc finger protein 627 (ZNF627);
the Asp51Ala A/C CC genotype in the gene encoding Interleukin 1 family, member 10 (ILIF10); or
the Asn159Asn A/G AG or GG genotype in the gene encoding Serpin 2;
   may be indicative of an increased risk of developing ACS, arterial inflammation, or ACS-associated impaired vascular function.

The methods of the invention are particularly useful in smokers (both current and former).

Where the following discussion refers to aspects of the invention useful to determine a subject's risk of developing ACS, it will be appreciated that these aspects of the invention are also useful in determining a subject's risk of developing ACS-associated impaired vascular function, and in determining a subject's risk of developing arterial inflammation.

It will be appreciated that the methods of the invention identify two categories of polymorphisms—namely those associated with a reduced risk of developing ACS, arterial inflammation, or ACS-associated impaired vascular function (which can be termed “protective polymorphisms”) and those associated with an increased risk of developing ACS, arterial inflammation, or ACS-associated impaired vascular function (which can be termed “susceptibility polymorphisms”).

Therefore, the present invention further provides a method of assessing a subject's risk of developing ACS, said method comprising:

determining the presence or absence of at least one protective polymorphism associated with a reduced risk of developing ACS; and

in the absence of at least one protective polymorphism, determining the presence or absence of at least one susceptibility polymorphism associated with an increased risk of developing ACS;

wherein the presence of one or more of said protective polymorphisms is indicative of a reduced risk of developing ACS, and the absence of at least one protective polymorphism in combination with the presence of at least one susceptibility polymorphism is indicative of an increased risk of developing ACS.

Again, it will be appreciated that the above aspect may be used to determine a subject's risk of developing ACS, arterial inflammation, or ACS-associated impaired vascular function.

Preferably, said at least one protective polymorphism is selected from the group consisting of:
the Asp92Asn A/G AA or AG genotype in the gene encoding Myeloid IgA Fc receptor (FCAR);
the A387P C/G GG genotype in the gene encoding Thrombospondin 4;
the A/G (rs4804611) AA genotype in the gene encoding Zinc finger protein 627 (ZNF627);
the Asn159Asn A/G AA genotype in the gene encoding Serpin 2; or
the C3279T A/G GG genotype in the gene encoding Galectin-2 (LGALS2).

The at least one susceptibility polymorphism may be selected from the group consisting of:
the Y402H C/T TT genotype in the gene encoding Complement Factor H;
the Asp92Asn A/G GG genotype in the gene encoding Myeloid IgA Fc receptor (FCAR);
the A/G (rs4804611) GA or GG genotype in the gene encoding Zinc finger protein 627 (ZNF627);
the Asp51Ala A/C CC genotype in the gene encoding Interleukin 1 family, member 10 (ILIF10); or
the Asn159Asn A/G AG or GG genotype in the gene encoding Serpin 2.

In a preferred form of the invention the presence of two or more protective polymorphisms is indicative of a reduced risk of developing ACS.

In a further preferred form of the invention the presence of two or more susceptibility polymorphisms is indicative of an increased risk of developing ACS, arterial inflammation, or ACS-associated impaired vascular function.

In still a further preferred form of the invention the presence of two or more protective polymorphisms irrespective of the presence of one or more susceptibility polymorphisms is indicative of reduced risk of developing ACS, arterial inflammation, or ACS-associated impaired vascular function.

In another aspect, the invention provides a method of determining a subject's risk of developing ACS, arterial inflammation, or ACS-associated impaired vascular function, said method comprising obtaining the result of one or more genetic tests of a sample from said subject, and analyzing the result for the presence or absence of one or more polymorphisms selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2;
C3279T A/G in the gene encoding Galectin-2 (LGALS2); or
one or more polymorphisms in linkage disequilibrium with any one or more of these polymorphisms;

wherein a result indicating the presence or absence of one or more of said polymorphisms is indicative of the subject's risk of developing ACS.

In a further aspect there is provided a method of determining a subject's risk of developing ACS comprising the analysis of two or more polymorphisms selected from the group consisting of:
Y402H C/T in the gene encoding Complement Factor H (CFH);
Asp92Asn A/G in the gene encoding Myeloid IgA Fc receptor (FCAR);
A387P C/G in the gene encoding Thrombospondin 4;
A/G (rs4804611) in the gene encoding Zinc finger protein 627 (ZNF627);
Asp51Ala A/C in the gene encoding Interleukin 1 family, member 10 (ILIF10);
Asn159Asn A/G in the gene encoding Serpin 2;
C3279T A/G in the gene encoding Galectin-2 (LGALS2); or
one or more polymorphisms in linkage disequilibrium with any one or more of these polymorphisms.

In various embodiments, any one or more of the above methods comprises the step of analyzing the amino acid present at a position mapping to codon 402 of the gene encoding CFH.

The presence of histidine at said position is indicative of a reduced risk of developing ACS.

The presence of tyrosine at said position is indicative of an increased risk of developing ACS.

In various embodiments, any one or more of the above methods comprises the step of analyzing the amino acid present at a position mapping to codon 92 of the gene encoding FCAR.

The presence of aspartic acid at said position is indicative of a decreased risk of developing ACS.

The presence of asparagine at said position is indicative of an increased risk of developing ACS.

In various embodiments, any one or more of the above methods comprises the step of analyzing the amino acid present at a position mapping to codon 387 of the gene encoding Thrombospondin 4.

The presence of alanine at said position is indicative of a decreased risk of developing ACS.

The presence of proline at said position is indicative of an increased risk of developing ACS.

In various embodiments, any one or more of the above methods comprises the step of analyzing the amino acid present at a position mapping to codon 51 of the gene encoding IL1F10.

The presence of aspartic acid at said position is indicative of a decreased risk of developing ACS.

The presence of alanine at said position may be indicative of an increased risk of developing ACS.

In a preferred form of the invention the methods as described herein are performed in conjunction with an analysis of one or more risk factors, including one or more epidemiological risk factors, associated with a risk of developing ACS. Such epidemiological risk factors include but are not limited to smoking or exposure to tobacco smoke, age, sex, and familial history of ACS.

In a further aspect, the invention provides for the use of at least one polymorphism in the assessment of a subject's risk of developing ACS, arterial inflammation, or ACS-associated impaired vascular function, wherein said at least one polymorphism is selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2;
C3279T A/G in the gene encoding Galectin-2 (LGALS2);
   one or more polymorphisms in linkage disequilibrium with any one of said polymorphisms.

Optionally, said use may be in conjunction with the use of at least one further polymorphism selected from the group consisting of:
A387P C/G in the gene encoding Thrombospondin 4;
Asp51Ala A/C in the gene encoding Interleukin 1 family, member 10 (ILIF10);
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2);
372 T/C in the gene encoding Tissue inhibitor of metalloproteinase 1 (TIMP1);
−509 C/T in the gene encoding Transforming growth factor β1 (TGFB1);
Thr26Asn A/C in the gene encoding Lymphotoxin α (LTA);
Asp299Gly A/G in the gene encoding Toll-like Receptor 4 (TLR4);
Thr399Ile C/T in the gene encoding TLR4;
−63 T/A in the gene encoding Nuclear factor of kappa light polypeptide gene enhancer in B-cells inhibitor-like 1 (NFKBIL1);
−1630 Ins/Del (AACTT/Del) in the gene encoding Platelet derived growth factor receptor alpha (PDGFRA);
1607 1G/2G (Del/G) in the gene encoding Matrix metalloproteinase 1 (MMP1);
12 IN 5 C/T in the gene encoding Platelet derived growth factor alpha (PDGFA);
−588 C/T in the gene encoding Glutamate-cysteine ligase modifier subunit (GCLM);
Ile132Val A/G in the gene encoding Olfactory receptor analogue OR13G1 (OR13G1);
Glu288Val A/T (M/S) in the gene encoding alpha 1-antitrypsin (α1-AT);
K469E A/G in the gene encoding Intracellular adhesion molecule 1 (ICAM1);
−23 C/G in the gene encoding HLA-B associated transcript 1 (BAT1);
Glu298Asp G/T in the gene encoding Nitric Oxide synthase 3 (NOS3);
−668 4G/5G in the gene encoding Plasminogen activator inhibitor 1 (PAI-1);
−181 A/G in the gene encoding Matrix metalloproteinase 7 (MMP7);
or one or more polymorphisms which are in linkage disequilibrium with any one or more of these polymorphisms.

In another aspect the invention provides a set of nucleotide probes and/or primers for use in the preferred methods of the invention herein described. Preferably, the nucleotide probes and/or primers are those which span, or are able to be used to span, the polymorphic regions of the genes. Also provided are one or more nucleotide probes and/or primers comprising the sequence of any one of the probes and/or primers herein described, including any one comprising the sequence of any one of SEQ. ID. NO. 1 to 35.

In yet a further aspect, the invention provides a nucleic acid microarray for use in the methods of the invention, which microarray comprises a substrate presenting nucleic acid sequences capable of hybridizing to nucleic acid sequences which encode one or more of the susceptibility or protective polymorphisms described herein or sequences complementary thereto.

In another aspect, the invention provides an antibody microarray for use in the methods of the invention, which microarray comprises a substrate presenting antibodies capable of binding to a product of expression of a gene the expression of which is upregulated or downregulated when associated with a susceptibility or protective polymorphism as described herein.

In a further aspect the present invention provides a method treating a subject having an increased risk of developing ACS, arterial inflammation, or ACS-associated impaired vascular function, the method comprising the step of replicating, genotypically or phenotypically, the presence and/or functional effect of a protective polymorphism as defined herein in said subject.

In yet a further aspect, the present invention provides a method of treating a subject having an increased risk of developing ACS, arterial inflammation, or ACS-associated impaired vascular function, said subject having a detectable susceptibility polymorphism as defined herein which either upregulates or downregulates expression of a gene such that the physiologically active concentration of the expressed gene product is outside a range which is normal for the age and sex of the subject, said method comprising the step of restoring the physiologically active concentration of said product of gene expression to be within a range which is normal for the age and sex of the subject.

In a further aspect the present invention provides a method of treating a subject having an increased risk of developing ACS due to the presence of a polymorphism predictive of susceptibility to ACS as defined herein comprising the step of reversing, genotypically or phenotypically, the functional effect of said polymorphism in said subject.

In yet a further aspect, the present invention provides a method for screening for compounds that modulate the expression and/or activity of a gene, the expression of which is upregulated or downregulated when associated with a susceptibility or protective polymorphism as defined herein (as compared to the level of expression of said gene when not associated with said polymorphism), said method comprising the steps of:

contacting a candidate compound with a cell comprising a susceptibility or protective polymorphism which has been determined to be associated with the upregulation or downregulation of expression of a gene; and

measuring the expression of said gene following contact with said candidate compound,

wherein a change in the level of expression after the contacting step as compared to before the contacting step is indicative of the ability of the compound to modulate the expression and/or activity of said gene.

Preferably, said cell is a human vascular cell, more preferably a human vascular epithelial cell, which has been pre-screened to confirm the presence of said polymorphism.

Preferably, said cell comprises a susceptibility polymorphism associated with upregulation of expression of said gene and said screening is for candidate compounds which downregulate expression of said gene.

Alternatively, said cell comprises a susceptibility polymorphism associated with downregulation of expression of said gene and said screening is for candidate compounds which upregulate expression of said gene.

In another embodiment, said cell comprises a protective polymorphism associated with upregulation of expression of said gene and said screening is for candidate compounds which further upregulate expression of said gene.

Alternatively, said cell comprises a protective polymorphism associated with downregulation of expression of said gene and said screening is for candidate compounds which further downregulate expression of said gene.

In another aspect, the present invention provides a method for screening for compounds that modulate the expression and/or activity of a gene, the expression of which is upregulated or downregulated when associated with a susceptibility or protective polymorphism as defined herein, said method comprising the steps of:

contacting a candidate compound with a cell comprising a gene, the expression of which is upregulated or downregulated when associated with a susceptibility or protective polymorphism but which in said cell the expression of which is neither upregulated nor downregulated; and

measuring the expression of said gene following contact with said candidate compound,

wherein a change in the level of expression after the contacting step as compared to before the contacting step is indicative of the ability of the compound to modulate the expression and/or activity of said gene.

Preferably, expression of the gene is downregulated when associated with a susceptibility polymorphism once said screening is for candidate compounds which in said cell, upregulate expression of said gene.

Preferably, said cell is a human vascular cell, more preferably a human vascular epithelial cell, which has been pre-screened to confirm the presence, and baseline level of expression, of said gene.

Alternatively, expression of the gene is upregulated when associated with a susceptibility polymorphism and said screening is for candidate compounds which, in said cell, downregulate expression of said gene.

In another embodiment, expression of the gene is upregulated when associated with a protective polymorphism and said screening is for compounds which, in said cell, upregulate expression of said gene.

Alternatively, expression of the gene is downregulated when associated with a protective polymorphism and said screening is for compounds which, in said cell, downregulate expression of said gene.

In yet a further aspect, the present invention provides a method of assessing the likely responsiveness of a subject at risk of developing or suffering from ACS to a prophylactic or therapeutic treatment, which treatment involves restoring the physiologically active concentration of a product of gene expression to be within a range which is normal for the age and sex of the subject, which method comprises detecting in said subject the presence or absence of a susceptibility polymorphism as defined herein which when present either upregulates or downregulates expression of said gene such that the physiological active concentration of the expressed gene product is outside said normal range, wherein the detection of the presence of said polymorphism is indicative of the subject likely responding to said treatment.

In still a further aspect, the present invention provides a method of assessing a subject's suitability for an intervention that is diagnostic of or therapeutic for ACS, the method comprising:
a) providing a net score for said subject, wherein the net score is or has been determined by:
i) providing the result of one or more genetic tests of a sample from the subject, and analyzing the result for the presence or absence of one or more protective polymorphisms or for the presence or absence of one or more susceptibility polymorphisms, wherein said protective or susceptibility polymorphisms are selected from the group consisting of:
Y402H C/T (rs1061170) in the gene encoding Complement Factor H (CFH);
Asp92Asn A/G (rs11666735) in the gene encoding Myeloid IgA Fc receptor (FCAR);
A/G (rs4804611) in the gene encoding Zinc finger protein 627 (ZNF627);
Asn159Asn A/G (rs6747096) in the gene encoding Serpin 2;
C3279T A/G (rs7291467) in the gene encoding Galectin-2 (LGALS2)
or one or more polymorphisms which are in linkage disequilibrium with any one or more of said polymorphisms;
ii) assigning a positive score for each protective polymorphism and a negative score for each susceptibility polymorphism or vice versa;
iii) calculating a net score for said subject by representing the balance between the combined value of the protective polymorphisms and the combined value of the susceptibility polymorphisms present in the subject sample;
and
b) providing a distribution of net scores for ACS sufferers and non-sufferers wherein the net scores for ACS sufferers and non-sufferers are or have been determined in the same manner as the net score determined for said subject;
c) determining whether the net score for said subject lies within a threshold on said distribution separating individuals deemed suitable for said intervention from those for whom said intervention is deemed unsuitable;
wherein a net score within said threshold is indicative of the subject's suitability for the intervention, and wherein a net score outside the threshold is indicative of the subject's unsuitability for the intervention.

The value assigned to each protective polymorphism may be the same or may be different. The value assigned to each susceptibility polymorphism may be the same or may be different, with either each protective polymorphism having a negative value and each susceptibility polymorphism having a positive value, or vice versa.

In one embodiment, the intervention is a diagnostic test for ACS.

In another embodiment, the intervention is a therapy for ACS, more preferably a preventative therapy for ACS.

Preferably, the one or more additional protective or susceptibility polymorphisms are selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2; or
C3279T A/G in the gene encoding Galectin-2 (LGALS2);
A387P C/G in the gene encoding Thrombospondin 4;
Asp51Ala A/C in the gene encoding Interleukin 1 family, member 10 (ILIF10);
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2);
372 T/C in the gene encoding Tissue inhibitor of metalloproteinase 1 (TIMP1);
−509 C/T in the gene encoding Transforming growth factor β1 (TGFB1);
Thr26Asn A/C in the gene encoding Lymphotoxin α (LTA);
Asp299Gly A/G in the gene encoding Toll-like Receptor 4 (TLR4);
Thr399Ile C/T in the gene encoding TLR4;
−63 T/A in the gene encoding Nuclear factor of kappa light polypeptide gene enhancer in B-cells inhibitor-like 1 (NFKBIL1);
−1630 Ins/Del (AACTT/Del) in the gene encoding Platelet derived growth factor receptor alpha (PDGFRA);
−1607 1G/2G (Del/G) in the gene encoding Matrix metalloproteinase 1 (MMP1);
12 IN 5 C/T in the gene encoding Platelet derived growth factor alpha (PDGFA);
−588 C/T in the gene encoding Glutamate-cysteine ligase modifier subunit (GCLM);
Ile132Val A/G in the gene encoding Olfactory receptor analogue OR13G1 (OR13G1);
Glu288Val A/T (M/S) in the gene encoding alpha 1-antitrypsin (α1-AT);
K469E A/G in the gene encoding Intracellular adhesion molecule 1 (ICAM1);
−23 C/G in the gene encoding HLA-B associated transcript 1 (BAT 1);
Glu298Asp G/T in the gene encoding Nitric Oxide synthase 3 (NOS3);
−668 4G/5G in the gene encoding Plasminogen activator inhibitor 1 (PAI-1);
−181 A/G in the gene encoding Matrix metalloproteinase 7 (MMP7);
or one or more polymorphisms which are in linkage disequilibrium with any one or more of these polymorphisms. More preferably, the protective and susceptibility polymorphisms are selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2;
C3279T A/G in the gene encoding Galectin-2 (LGALS2);
or one or more polymorphisms in linkage disequilibrium with one or more of said polymorphisms.

In a still further aspect, the invention provides for the use of data predictive of the predisposition of a subject to ACS, arterial inflammation, or ACS-associated impaired vascular function in the determination of the subject's suitability for an intervention that is diagnostic of or therapeutic for ACS, arterial inflammation, or ACS-associated impaired vascular function,

said data comprising, consisting of or including the result of at least one ACS-associated genetic analysis selected from one or more of the genetic analyses described herein and/or the Cardiogene™-brand cardiovascular test,

and said data being indicative of the subject's suitability or unsuitability for the intervention.

In one embodiment the data is a net score determined as described above.

In another embodiment, the data is representative of whether the net score for a subject lies within a threshold on said distribution separating individuals deemed suitable for said intervention from those for whom said intervention is deemed unsuitable.

In another aspect, the invention provides a system for determining a subject's risk of developing ACS, arterial inflammation, or ACS-associated impaired vascular function, said system comprising:

computer processor means for receiving, processing and communicating data;

storage means for storing data including a reference genetic database of the results of at least one genetic analysis with respect to ACS, arterial inflammation, or ACS-associated impaired vascular function and optionally a reference non-genetic database of non-genetic risk factors for ACS; and

a computer program embedded within the computer processor which, once data consisting of or including the result of a genetic analysis for which data is included in the reference genetic database is received, processes said data in the context of said reference databases to determine, as an outcome, the subject's risk of developing ACS, arterial inflammation, or ACS-associated impaired vascular function, said outcome being communicable once known, preferably to a user having input said data.

Preferably, the at least one genetic analysis is an analysis of one or more polymorphisms selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2;
C3279T A/G in the gene encoding Galectin-2 (LGALS2); or
one or more polymorphisms which are in linkage disequilibrium with said one or more polymorphisms.

In one embodiment, the data is input by a representative of a healthcare provider.

In another embodiment, the data is input by the subject, their medical advisor or other representative.

Preferably, said system is accessible via the internet or by personal computer.

Preferably, said reference genetic database consists of, comprises or includes the results of an ACS-associated genetic analysis selected from one or more of the genetic analyses described herein and/or the Cardiogene™-brand cardiovascular test, preferably the results of an analysis of one or more polymorphisms selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2;
C3279T A/G in the gene encoding Galectin-2 (LGALS2); or
one or more polymorphisms which are in linkage disequilibrium with said one or more polymorphisms.

More preferably, said reference genetic database consists of, comprises or includes the results of an analysis of any two, any three, any four, or all of the polymorphisms selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2; or
C3279T A/G in the gene encoding Galectin-2 (LGALS2).

The reference genetic database may additionally comprise or include the results of an analysis of one or more further polymorphisms selected from the group consisting of:
A387P C/G in the gene encoding Thrombospondin 4; or
Asp51Ala A/C in the gene encoding Interleukin 1 family, member 10 (ILIF10).

More preferably, said reference genetic database consists of, comprises or includes the results of all of the genetic analyses described herein and the Cardiogene™-brand cardiovascular test.

In yet a further aspect, the invention provides a computer program suitable for use in a system as defined above comprising a computer usable medium having program code embodied in the medium for causing the computer program to process received data consisting of or including the result of at least one ACS-associated genetic analysis in the context of both a reference genetic database of the results of said at least one ACS-associated genetic analysis and optionally a reference non-genetic database of non-genetic risk factors for ACS.

Preferably, the at least one ACS-associated genetic analysis is selected from one or more of the genetic analyses described herein and/or the Cardiogene™-brand cardiovascular test, preferably the at least one ACS-associated genetic analysis is an analysis of one or more polymorphisms selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2;
C3279T A/G in the gene encoding Galectin-2 (LGALS2); or
one or more polymorphisms which are in linkage disequilibrium with said one or more polymorphisms.

Preferably, the at least one ACS-associated genetic analysis is an analysis of any two, any three, any four, or all of the polymorphisms selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2; or
C3279T A/G in the gene encoding Galectin-2 (LGALS2).

The at least one ACS-associated genetic analysis can additionally comprise the analysis of one or more further polymorphisms selected from the group consisting of:
A387P C/G in the gene encoding Thrombospondin 4; or
Asp51Ala A/C in the gene encoding Interleukin 1 family, member 10 (ILIF10).

Preferably, the at least one ACS-associated genetic analysis is an analysis of the genetic analyses described herein and the Cardiogene™-brand cardiovascular test.

Also provided are computer systems and programs as described above for the determination of the subject's suitability for an intervention that is diagnostic of or therapeutic for ACS.

In a still further aspect, the invention provides for the use of data predictive of the predisposition of a subject to ACS, arterial inflammation, or ACS-associated impaired vascular function in the determination of the subject's risk of developing ACS, arterial inflammation, or ACS-associated impaired vascular function,

said data comprising, consisting of or including the result of at least one ACS-associated genetic analysis selected from one or more of the genetic analyses described herein and/or the Cardiogene™-brand cardiovascular test,

and said data being representative of the subject's risk of developing ACS, arterial inflammation, or ACS-associated impaired vascular function.

Preferably, the data comprises, consists of or includes the result of an analysis of one or more polymorphisms selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2;
C3279T A/G in the gene encoding Galectin-2 (LGALS2); or
one or more polymorphisms which are in linkage disequilibrium with said one or more polymorphisms

More preferably, the data comprises, consists of or includes the results of an analysis of two or more, three or more, four or more, or all of the above polymorphisms.

More preferably, the data comprises, consists of or includes the results of all of the genetic analyses described herein and the Cardiogene™-brand cardiovascular test.

In a further aspect, the present invention provides a kit for assessing a subject's risk of developing ACS, arterial inflammation, or ACS-associated impaired vascular function, said kit comprising a means of analyzing a sample from said subject for the presence or absence of one or more polymorphisms disclosed herein.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### Description of the Preferred Embodiments

Using case-control studies the frequencies of several genetic variants (polymorphisms) of candidate genes in smokers who have developed ACS and blood donor controls have been compared. The majority of these candidate genes have confirmed (or likely) functional effects on gene expression or protein function. Specifically, the frequencies of polymorphisms between resistant smokers and those with ACS have been compared.

In one embodiment described herein 5 susceptibility genetic polymorphisms and 5 protective genetic polymorphisms are identified. These are as follows:

| | | | | |
|---|---|---|---|---|
| Gene | Polymorphism | Rs# | Genotype | Phenotype |

| | | | | |
|---|---|---|---|---|
| CFH | Y402 H | 1061170 | TT | susceptibility |
| FCAR (IgA Fc receptor) | As92Asn | 11666735 | AA/AG | protective |
| | | | GG | (susceptibility) |
| Thrombospondin 4 | A387P | 1866389 | GG | protective |
| ZNF627 | A/G | 4804611 | GA/GG | susceptibility |
| | | | AA | (protective) |
| IL1F10 | Asp51Ala | 6743376 | CC | susceptibility |
| Serpin 2 | Asn159Asn | 6747096 | AG/GG | susceptibility |
| | | | AA | (protective) |
| Galectin-2 (LGALS2) | C3279T | 7291467 | GG | protective |

A susceptibility genetic polymorphism (also referred to herein as a susceptibility polymorphism) is one which, when present, is indicative of an increased risk of developing ACS. In contrast, a protective genetic polymorphism (also referred to herein as a protective polymorphism) is one which, when present, is indicative of a reduced risk of developing ACS.

As used herein, the phrase “risk of developing ACS” means the likelihood that a subject to whom the risk applies will develop ACS, and includes predisposition to, and potential onset of the disease. Accordingly, the phrase “increased risk of developing ACS” means that a subject having such an increased risk possesses an hereditary inclination or tendency to develop ACS. This does not mean that such a person will actually develop ACS at any time, merely that he or she has a greater likelihood of developing ACS compared to the general population of individuals that either does not possess a polymorphism associated with increased ACS or does possess a polymorphism associated with decreased ACS risk. Subjects with an increased risk of developing ACS include those with a predisposition to ACS, such as a tendency or predilection regardless of their vascular function at the time of assessment, for example, a subject who is genetically inclined to ACS but who has normal vascular function, those at potential risk, including subjects with a tendency to mildly reduced vascular function who are likely to go on to suffer ACS if they keep smoking, and subjects with potential onset of ACS, who have a tendency to poor vascular function consistent with ACS at the time of assessment.

Similarly, the phrase “decreased risk of developing ACS” means that a subject having such a decreased risk possesses an hereditary disinclination or reduced tendency to develop ACS. This does not mean that such a person will not develop ACS at any time, merely that he or she has a decreased likelihood of developing ACS compared to the general population of individuals that either does possess one or more polymorphisms associated with increased ACS, or does not possess a polymorphism associated with decreased ACS.

It will therefore be apparent that the phrase “risk of developing ACS, arterial inflammation, or ACS-associated impaired vascular function” means the likelihood that a subject to whom the risk applies will develop ACS, arterial inflammation, or ACS-associated impaired vascular function, and includes predisposition to, and potential onset of the disease or condition.

It will be understood that in the context of the present invention the term “polymorphism” means the occurrence together in the same population at a rate greater than that attributable to random mutation (usually greater than 1%) of two or more alternate forms (such as alleles or genetic markers) of a chromosomal locus that differ in nucleotide sequence or have variable numbers of repeated nucleotide units. See www.ornl.gov/sci/techresources/Human_Genome/publicat/97pr/09gloss.html#p. Accordingly, the term “polymorphisms” is used herein contemplates genetic variations, including single nucleotide substitutions, insertions and deletions of nucleotides, repetitive sequences (such as microsatellites), and the total or partial absence of genes (eg. null mutations). As used herein, the term “polymorphisms” also includes genotypes and haplotypes. A genotype is the genetic composition at a specific locus or set of loci. A haplotype is a set of closely linked genetic markers present on one chromosome which are not easily separable by recombination, tend to be inherited together, and may be in linkage disequilibrium. A haplotype can be identified by patterns of polymorphisms such as SNPs. Similarly, the term “single nucleotide polymorphism” or “SNP” in the context of the present invention includes single base nucleotide substitutions and short deletion and insertion polymorphisms.

A reduced or increased risk of a subject developing ACS may be diagnosed by analyzing a sample from said subject for the presence of a polymorphism selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2;
C3279T A/G in the gene encoding Galectin-2 (LGALS2); or
one or more polymorphisms which are in linkage disequilibrium with any one or more of the above group.

These polymorphisms can also be analyzed in combinations of two or more, or in combination with other polymorphisms indicative of a subject's risk of developing ACS, inclusive of the remaining polymorphisms listed above. In particular, these polymorphisms can be analyzed in combination with one or more polymorphisms selected from the group consisting of:
A387P C/G in the gene encoding Thrombospondin 4; or
Asp51Ala A/C in the gene encoding Interleukin 1 family, member 10 (ILIF10).

Assays which involve combinations of polymorphisms, including those amenable to high throughput, such as those utilizing microarrays, are preferred.

Statistical analyses, particularly of the combined effects of these polymorphisms, show that the genetic assays of the present invention can be used to determine the risk quotient of any subject (including smokers) and in particular to identify subjects at greater risk of developing ACS. Such combined analysis can be of combinations of susceptibility polymorphisms only, of protective polymorphisms only, or of combinations of both. Analysis can also be step-wise, with analysis of the presence or absence of protective polymorphisms occurring first and then with analysis of susceptibility polymorphisms proceeding only where no protective polymorphisms are present.

Thus, through systematic analysis of the frequency of these polymorphisms in well defined groups of subjects including smokers and non-smokers as described herein, it is possible to implicate certain genes and proteins in the development of ACS and improve the ability to identify which subjects are at increased risk of developing ACS-related impaired vascular function, arterial inflammation, and ACS for predictive purposes.

Acute Coronary Syndrome

Acute coronary syndrome (“ACS”) is a complex disorder which has been variously defined. See, for example, U.S. Pat. No. 6,706,689, wherein ACS denotes subjects who have or are at high risk of developing an acute myocardial infarction (MI), and includes unstable angina (UA), non-Q-wave cardiac necrosis (NQCN) and Q-wave MI (QMI). As described therein, ACS is typically diagnosed when a patient has acute (i.e., sudden onset) chest pain of a cardiac origin that is either new or clearly different from pre-existing, chronic, stable angina; that is, ACS chest pain is more severe, more frequent, occurs at rest, or is longer than 15 minutes in duration. After ACS has been diagnosed, the patient is stratified into UA, NQCN, and QMI, using criteria set forth in U.S. Pat. No. 6,706,689. As described therein, Q-wave MI generally is understood to result from total occlusion of a coronary artery, whereas UA is caused by a subtotal occlusion. Again as described in U.S. Pat. No. 6,706,689, a number of clinical indicators that aid a diagnosis of ACS are known including elevated troponin 1 levels, elevated troponin T levels, elevated CK-MB levels, and elevated LDH, LDH1 and LDH2 levels.

Local and systemic inflammatory processes, including pro-inflammatory cytokine generation and release and localization and activation of inflammatory cells including foam cells, macrophages, lymphocytes, and mast cells are associated with arterial inflammation and have been implicated in the pathogenesis of ACS (See Mulvihill N T and Foley J B, 2001), and are believed to play a significant pathophysiologic role in coronary plaque disruption. Plaque disruption in turn leads to inter alia platelet aggregation and thrombosis. It is recognized that thrombosis underlies most acute complications of atherosclerosis, notably unstable angina and acute myocardial infarction.

Accordingly, the methods of the present invention are suitable for the identification of subject's risk of developing arterial inflammation comprising analyzing a sample from said subject for the presence or absence of one or more polymorphisms selected from the group consisting of:
Y402H C/T (rs1061170) in the gene encoding Complement Factor H (CFH);
Asp92Asn A/G (rs11666735) in the gene encoding Myeloid IgA Fc receptor (FCAR);
A/G (rs4804611) in the gene encoding Zinc finger protein 627 (ZNF627);
Asn159Asn A/G (rs6747096) in the gene encoding Serpin 2; or
C3279T A/G (rs7291467) in the gene encoding Galectin-2 (LGALS2);

wherein the presence or absence of one or more of said polymorphisms is indicative of the subject's risk of developing arterial inflammation.

Preferably, the arterial inflammation is coronary artery inflammation.

The method can additionally comprise analyzing a sample from said subject for the presence of one or more further polymorphisms selected from the group consisting of:
A387P C/G (rs1866389) in the gene encoding Thrombospondin 4; or

Asp51Ala A/C (rs6743376) in the gene encoding Interleukin 1 family, member 10 (ILIF10). The invention is also useful in determining a subject's risk of developing ACS-associated impaired vascular function, which may be evident before diagnosable ACS is evident. As used herein, the phrase “ACS-associated impaired vascular function” contemplates ischemia, vasoconstriction, coronary spasm, erosion, occlusion, plaque rupture, impaired platelet aggregation, and the like. Although it perhaps represents ACS-associated impaired vascular function in extremis, thrombosis per se will typically be considered evidentiary of ACS, rather than impaired vascular function.

The present results show that the minority of smokers who develop ACS do so because they have one or more of the susceptibility polymorphisms and few or none of the protective polymorphisms defined herein. It is thought that the presence of one or more susceptibility polymorphisms, together with the damaging irritant and oxidant effects of smoking, combine to make this group of smokers highly susceptible to developing ACS. Additional risk factors, such as familial history, age, weight, pack years, etc., will also have an impact on the risk profile of a subject, and can be assessed in combination with the genetic analyses described herein. The one or more polymorphisms can be detected directly or by detection of one or more polymorphisms which are in linkage disequilibrium with said one or more polymorphisms. As discussed above, linkage disequilibrium is a phenomenon in genetics whereby two or more mutations or polymorphisms are in such close genetic proximity that they are co-inherited. This means that in genotyping, detection of one polymorphism as present infers the presence of the other. (Reich DE et al; Linkage disequilibrium in the human genome, Nature 2001, 411:199-204.) Various degrees of linkage disequilibrium are possible. Preferably, the one or more polymorphisms in linkage disequilibrium with one or more of the polymorphisms specified herein are in greater than about 60% linkage disequilibrium, are in about 70% linkage disequilibrium, about 75%, about 80%, about 85%, about 90%, about 91%, 92%, 93%, 94%, 95%, 96%, 97%, 98%, 99%, or about 100% linkage disequilibrium with one or more of the polymorphisms specified herein. Those skilled in the art will appreciate that linkage disequilibrium may also, when expressed with reference to the deviation of the observed frequency of a pair of alleles from the expected, be denoted by a capital D. Accordingly, the phrase “two alleles are in LD” usually means that D does not equal 0. Contrariwise, “linkage equilibrium” denotes the case D=0. When utilising this nomenclature, the one or more polymorphisms in LD with the one or more polymorphisms specified herein are preferably in LD of greater than about D′=0.6, of about D′=0.7, of about D′=0.75, of about D′=0.8, of about D′=0.85, of about D′=0.9, of about D′=0.91, of about D′=0.92, of about D′=0.93, of about D′=0.94, of about D′=0.95, of about D′=0.96, of about D′=0.97, of about D′=0.98, of about D′=0.99, or about D′=1.0. (Devlin and Risch 1995; A comparison of linkage disequilibrium measures for fine-scale mapping, Genomics 29: 311-322).

It will be apparent that polymorphsisms in linkage disequilibrium with one or more other polymorphism associated with increased or decreased risk of developing ACS will also provide utility as biomarkers for risk of developing ACS. The frequency for SNPs in linkage disequilibrium are often very similar. Accordingly, these genetically linked SNPs can be utilized in combined polymorphism analyses to derive a level of risk comparable to that calculated from the original SNP. An example of such an analysis in which SNPs in LD are substituted one for the other is presented in Example 2 of the Applicant's PCT International application PCT/NZ2006/000292, filed Nov. 10, 2006, which is incorporated herein by reference in its entirety.

It will therefore be apparent that one or more polymorphisms in linkage disequilibrium with the polymorphisms specified herein can be identified, for example, using public data bases. Examples of such polymorphisms reported to be in linkage disequilibrium with the polymorphisms specified herein are presented herein in Table 9.

It will also be apparent that frequently a variety of nomenclatures may exist for any given polymorphism. For example, the polymorphism referred to as Arg 213 Gly in the gene encoding SOD3 is believed to have been referred to variously as Arg 312 Gln, +760 G/C, and Arg 231 Gly (rs1799895). When referring to a susceptibility or protective polymorphism as herein described, such alternative nomenclatures are also contemplated by the present invention. Generally, such alternative nomenclatures can be readily identified by investigating for example the Genbank database using the unique identifier (e.g., the rs number) for a particular SNP.

Identification and Analysis of Polymorphisms

The methods of the invention are primarily directed to the detection and identification of the above polymorphisms associated with ACS. These polymorphisms are typically single nucleotide polymorphisms. In general terms, a single nucleotide polymorphism (SNP) is a single base change or point mutation resulting in genetic variation between individuals. SNPs occur in the human genome approximately once every 100 to 300 bases, and can occur in coding or non-coding regions. Due to the redundancy of the genetic code, a SNP in the coding region may or may not change the amino acid sequence of a protein product. A SNP in a non-coding region can, for example, alter gene expression by, for example, modifying control regions such as promoters, transcription factor binding sites, processing sites, ribosomal binding sites, and affect gene transcription, processing, and translation.

SNPs can facilitate large-scale association genetics studies, and there has recently been great interest in SNP discovery and detection. SNPs show great promise as markers for a number of phenotypic traits (including latent traits), such as for example, disease propensity and severity, wellness propensity, and drug responsiveness including, for example, susceptibility to adverse drug reactions. Knowledge of the association of a particular SNP with a phenotypic trait, coupled with the knowledge of whether an individual has said particular SNP, can enable the targeting of diagnostic, preventative and therapeutic applications to allow better disease management, to enhance understanding of disease states and to ultimately facilitate the discovery of more effective treatments, such as personalized treatment regimens.

Indeed, a number of databases have been constructed of known SNPs, and for some such SNPs, the biological effect associated with a SNP. For example, the NCBI SNP database “dbSNP” is incorporated into NCBI's Entrez system and can be queried using the same approach as the other Entrez databases such as PubMed and GenBank. This database has records for over 1.5 million SNPs mapped onto the human genome sequence. Each dbSNP entry includes the sequence context of the polymorphism (i.e., the surrounding sequence), the occurrence frequency of the polymorphism (by population or individual), and the experimental method(s), protocols, and conditions used to assay the variation, and can include information associating a SNP with a particular phenotypic trait.

At least in part because of the potential impact on health and wellness, there has been and continues to be a great deal of effort to develop methods that reliably and rapidly identify SNPs. This is no trivial task, at least in part because of the complexity of human genomic DNA, with a haploid genome of 3×10⁹ base pairs, and the associated sensitivity and discriminatory requirements.

Genotyping approaches to detect SNPs well-known in the art include DNA sequencing, methods that require allele specific hybridization of primers or probes, allele specific incorporation of nucleotides to primers bound close to or adjacent to the polymorphisms (often referred to as “single base extension”, or “minisequencing”), allele-specific ligation (joining) of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation is also possible where the SNP lies in a coding region and results in an amino acid change.

DNA sequencing allows the direct determination and identification of SNPs. The benefits in specificity and accuracy are generally outweighed for screening purposes by the difficulties inherent in whole genome, or even targeted subgenome, sequencing.

Mini-sequencing involves allowing a primer to hybridize to the DNA sequence adjacent to the SNP site on the test sample under investigation. The primer is extended by one nucleotide using all four differentially tagged fluorescent dideoxynucleotides (A,C,G, or T), and a DNA polymerase. Only one of the four nucleotides (homozygous case) or two of the four nucleotides (heterozygous case) is incorporated. The base that is incorporated is complementary to the nucleotide at the SNP position.

A number of methods currently used for SNP detection involve site-specific and/or allele-specific hybridization. These methods are largely reliant on the discriminatory binding of oligonucleotides to target sequences containing the SNP of interest. The techniques of Affymetrix (Santa Clara, Calif.) and Nanogen Inc. (San Diego, Calif.) are particularly well-known, and utilize the fact that DNA duplexes containing single base mismatches are much less stable than duplexes that are perfectly base-paired. The presence of a matched duplex is detected by fluorescence.

The majority of methods to detect or identify SNPs by site-specific hybridization require target amplification by methods such as PCR to increase sensitivity and specificity (see, for example U.S. Pat. No. 5,679,524, PCT publication WO 98/59066, PCT publication WO 95/12607). US Application 20050059030 (incorporated herein in its entirety) describes a method for detecting a single nucleotide polymorphism in total human DNA without prior amplification or complexity reduction to selectively enrich for the target sequence, and without the aid of any enzymatic reaction. The method utilizes a single-step hybridization involving two hybridization events: hybridization of a first portion of the target sequence to a capture probe, and hybridization of a second portion of said target sequence to a detection probe. Both hybridization events happen in the same reaction, and the order in which hybridization occurs is not critical.

US Application 20050042608 (incorporated herein in its entirety) describes a modification of the method of electrochemical detection of nucleic acid hybridization of Thorp et al. (U.S. Pat. No. 5,871,918). Briefly, capture probes are designed, each of which has a different SNP base and a sequence of probe bases on each side of the SNP base. The probe bases are complementary to the corresponding target sequence adjacent to the SNP site. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. These differences in the oxidation rates at the different electrodes are used to determine whether the selected nucleic acid target has a single nucleotide polymorphism at the selected SNP site.

The technique of Lynx Therapeutics (Hayward, Calif.) using MEGATYPE™ technology can genotype very large numbers of SNPs simultaneously from small or large pools of genomic material. This technology uses fluorescently labeled probes and compares the collected genomes of two populations, enabling detection and recovery of DNA fragments spanning SNPs that distinguish the two populations, without requiring prior SNP mapping or knowledge.

A number of other methods for detecting and identifying SNPs exist. These include the use of mass spectrometry, for example, to measure probes that hybridize to the SNP. This technique varies in how rapidly it can be performed, from a few samples per day to a high throughput of 40,000 SNPs per day, using mass code tags. A preferred example is the use of mass spectrometric determination of a nucleic acid sequence which comprises the polymorphisms of the invention, for example, which includes the promoter of the COX2 gene or a complementary sequence. Such mass spectrometric methods are known to those skilled in the art, and the genotyping methods of the invention are amenable to adaptation for the mass spectrometric detection of the polymorphisms of the invention, for example, the COX2 promoter polymorphisms of the invention.

SNPs can also be determined by ligation-bit analysis. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174.

U.S. Pat. No. 6,821,733 (incorporated herein in its entirety) describes methods to detect differences in the sequence of two nucleic acid molecules that includes the steps of: contacting two nucleic acids under conditions that allow the formation of a four-way complex and branch migration; contacting the four-way complex with a tracer molecule and a detection molecule under conditions in which the detection molecule is capable of binding the tracer molecule or the four-way complex; and determining binding of the tracer molecule to the detection molecule before and after exposure to the four-way complex. Competition of the four-way complex with the tracer molecule for binding to the detection molecule indicates a difference between the two nucleic acids.

Protein- and proteomics-based approaches are also suitable for polymorphism detection and analysis. Polymorphisms which result in or are associated with variation in expressed proteins can be detected directly by analyzing said proteins. This typically requires separation of the various proteins within a sample, by, for example, gel electrophoresis or HPLC, and identification of said proteins or peptides derived therefrom, for example by NMR or protein sequencing such as chemical sequencing or more prevalently mass spectrometry. Proteomic methodologies are well known in the art, and have great potential for automation. For example, integrated systems, such as the ProteomIQ™ system from Proteome Systems, provide high throughput platforms for proteome analysis combining sample preparation, protein separation, image acquisition and analysis, protein processing, mass spectrometry and bioinformatics technologies.

The majority of proteomic methods of protein identification utilize mass spectrometry, including ion trap mass spectrometry, liquid chromatography (LC) and LC/MSn mass spectrometry, gas chromatography (GC) mass spectroscopy, Fourier transform-ion cyclotron resonance-mass spectrometer (FT-MS), MALDI-TOF mass spectrometry, and ESI mass spectrometry, and their derivatives. Mass spectrometric methods are also useful in the determination of post-translational modification of proteins, such as phosphorylation or glycosylation, and thus have utility in determining polymorphisms that result in or are associated with variation in post-translational modifications of proteins.

Associated technologies are also well known, and include, for example, protein processing devices such as the “Chemical Inkjet Printer” comprising piezoelectric printing technology that allows in situ enzymatic or chemical digestion of protein samples electroblotted from 2-D PAGE gels to membranes by jetting the enzyme or chemical directly onto the selected protein spots. After in-situ digestion and incubation of the proteins, the membrane can be placed directly into the mass spectrometer for peptide analysis.

A large number of methods reliant on the conformational variability of nucleic acids have been developed to detect SNPs.

For example, Single Strand Conformational Polymorphism (SSCP, Orita et al, PNAS 1989 86:2766-2770) is a method reliant on the ability of single-stranded nucleic acids to form secondary structure in solution under certain conditions. The secondary structure depends on the base composition and can be altered by a single nucleotide substitution, causing differences in electrophoretic mobility under nondenaturing conditions. The various polymorphs are typically detected by autoradiography when radioactively labelled, by silver staining of bands, by hybridization with detectably labelled probe fragments or the use of fluorescent PCR primers which are subsequently detected, for example by an automated DNA sequencer.

Modifications of SSCP are well known in the art, and include the use of differing gel running conditions, such as for example differing temperature, or the addition of additives, and different gel matrices. Other variations on SSCP are well known to the skilled artisan, including, RNA-SSCP, restriction endonuclease fingerprinting-SSCP, dideoxy fingerprinting (a hybrid between dideoxy sequencing and SSCP), bidirectional dideoxy fingerprinting (in which the dideoxy termination reaction is performed simultaneously with two opposing primers), and Fluorescent PCR-SSCP (in which PCR products are internally labelled with multiple fluorescent dyes, may be digested with restriction enzymes, followed by SSCP, and analyzed on an automated DNA sequencer able to detect the fluorescent dyes).

Other methods which utilize the varying mobility of different nucleic acid structures include Denaturing Gradient Gel Electrophoresis (DGGE), Temperature Gradient Gel Electrophoresis (TGGE), and Heteroduplex Analysis (HET). Here, variation in the dissociation of double stranded DNA (for example, due to base-pair mismatches) results in a change in electrophoretic mobility. These mobility shifts are used to detect nucleotide variations.

Denaturing High Pressure Liquid Chromatography (HPLC) is yet a further method utilized to detect SNPs, using HPLC methods well-known in the art as an alternative to the separation methods described above (such as gel electrophoresis) to detect, for example, homoduplexes and heteroduplexes which elute from the HPLC column at different rates, thereby enabling detection of mismatch nucleotides and thus SNPs.

Yet further methods to detect SNPs rely on the differing susceptibility of single stranded and double stranded nucleic acids to cleavage by various agents, including chemical cleavage agents and nucleolytic enzymes. For example, cleavage of mismatches within RNA:DNA heteroduplexes by RNase A, of heteroduplexes by, for example bacteriophage T4 endonuclease YII or T7 endonuclease I, of the 5′ end of the hairpin loops at the junction between single stranded and double stranded DNA by cleavase I, and the modification of mispaired nucleotides within heteroduplexes by chemical agents commonly used in Maxam-Gilbert sequencing chemistry, are all well known in the art.

Further examples include the Protein Translation Test (PTT), used to resolve stop codons generated by variations which lead to a premature termination of translation and to protein products of reduced size, and the use of mismatch binding proteins. Variations are detected by binding of, for example, the MutS protein, a component of Escherichia coli DNA mismatch repair system, or the human hMSH2 and GTBP proteins, to double stranded DNA heteroduplexes containing mismatched bases. DNA duplexes are then incubated with the mismatch binding protein, and variations are detected by mobility shift assay. For example, a simple assay is based on the fact that the binding of the mismatch binding protein to the heteroduplex protects the heteroduplex from exonuclease degradation.

Those skilled in the art will know that a particular SNP, particularly when it occurs in a regulatory region of a gene such as a promoter, can be associated with altered expression of a gene. Altered expression of a gene can also result when the SNP is located in the coding region of a protein-encoding gene, for example where the SNP is associated with codons of varying usage and thus with tRNAs of differing abundance. Such altered expression can be determined by methods well known in the art, and can thereby be employed to detect such SNPs. Similarly, where a SNP occurs in the coding region of a gene and results in a non-synonomous amino acid substitution, such substitution can result in a change in the function of the gene product. Similarly, in cases where the gene product is an RNA, such SNPs can result in a change of function in the RNA gene product. Any such change in function, for example as assessed in an activity or functionality assay, can be employed to detect such SNPs.

The above methods of detecting and identifying SNPs are amenable to use in the methods of the invention.

Of course, in order to detect and identify SNPs in accordance with the invention, a sample containing material to be tested is obtained from the subject. The sample can be any sample potentially containing the target SNPs (or target polypeptides, as the case may be) and obtained from any bodily fluid (blood, urine, saliva, etc) biopsies or other tissue preparations.

DNA or RNA can be isolated from the sample according to any of a number of methods well known in the art. For example, methods of purification of nucleic acids are described in Tijssen; Laboratory Techniques in Biochemistry and Molecular Biology: Hybridization with nucleic acid probes Part 1: Theory and Nucleic acid preparation, Elsevier, New York, N.Y. 1993, as well as in Maniatis, T., Fritsch, E. F. and Sambrook, J., Molecular Cloning Manual 1989.

To assist with detecting the presence or absence of polymorphisms/SNPs, nucleic acid probes and/or primers can be provided. Such probes and/or primers have nucleic acid sequences specific for chromosomal changes evidencing the presence or absence of the polymorphism and are preferably labeled with a substance that emits a detectable signal when combined with the target polymorphism.

The nucleic acid probes and/or primers can be genomic DNA or cDNA or mRNA, or any RNA-like or DNA-like material, such as peptide nucleic acids, branched DNAs, and the like. The probes can be sense or antisense polynucleotide probes. Where target polynucleotides are double-stranded, the probes may be either sense or antisense strands. Where the target polynucleotides are single-stranded, the probes are complementary single strands.

The probes and/or primers can be prepared by a variety of synthetic or enzymatic schemes, which are well known in the art. The probes and/or primers can be synthesized, in whole or in part, using chemical methods well known in the art (Caruthers et al., Nucleic Acids Res., Symp. Ser., 215-233 (1980)). Alternatively, the probes can be generated, in whole or in part, enzymatically.

Nucleotide analogs can be incorporated into probes and/or primers by methods well known in the art. The only requirement is that the incorporated nucleotide analog must serve to base pair with target polynucleotide sequences. For example, certain guanine nucleotides can be substituted with hypoxanthine, which base pairs with cytosine residues. However, these base pairs are less stable than those between guanine and cytosine. Alternatively, adenine nucleotides can be substituted with 2,6-diaminopurine, which can form stronger base pairs than those between adenine and thymidine.

Additionally, the probes and/or primers can include nucleotides that have been derivatized chemically or enzymatically. Typical chemical modifications include derivatization with acyl, alkyl, aryl or amino groups.

The probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

Nucleic acid primers suitable for detecting the presence or absence of polymorphisms may be designed and synthesized by methods well known in the art. For example, primers suitable for primer extension and/or sequencing may be designed to bind immediately upstream of the polymorphic site, so that when extended the identity of the nucleotide at the polymorphic site is determined. Such primers are exemplary of primers that are able to be used to span the polymorphic region of the genes described herein, and specific examples of such primers are described herein (see for example Tables 2.1 and 2.3). Primers suitable for use in other detection methods well known in the art, for example PCR, TAQMAN, RTPCR and the like, are also contemplated.

Nucleic acid microarrays are preferred. Such microarrays (including nucleic acid chips) are well known in the art (see, for example U.S. Pat. Nos. 5,578,832; 5,861,242; 6,183,698; 6,287,850; 6,291,183; 6,297,018; 6,306,643; and 6,308,170, each incorporated by reference).

Alternatively, antibody microarrays can be produced. The production of such microarrays is essentially as described in Schweitzer & Kingsmore, “Measuring proteins on microarrays”, Curr Opin Biotechnol 2002; 13(1): 14-9; Avseekno et al., “Immobilization of proteins in immunochemical microarrays fabricated by electrospray deposition”, Anal Chem 200115; 73(24): 6047-52; Huang, “Detection of multiple proteins in an antibody-based protein microarray system, Immunol Methods 20011; 255 (1-2): 1-13.

The present invention also contemplates the preparation of kits for use in accordance with the present invention. Suitable kits include various reagents for use in accordance with the present invention in suitable containers and packaging materials, including tubes, vials, and shrink-wrapped and blow-molded packages.

Materials suitable for inclusion in an exemplary kit in accordance with the present invention comprise one or more of the following: gene specific PCR primer pairs (oligonucleotides) that anneal to DNA or cDNA sequence domains that flank the genetic polymorphisms of interest, reagents capable of amplifying a specific sequence domain in either genomic DNA or cDNA without the requirement of performing PCR; reagents required to discriminate between the various possible alleles in the sequence domains amplified by PCR or non-PCR amplification (e.g., restriction endonucleases, oligonucleotide that anneal preferentially to one allele of the polymorphism, including those modified to contain enzymes or fluorescent chemical groups that amplify the signal from the oligonucleotide and make discrimination of alleles more robust); reagents required to physically separate products derived from the various alleles (e.g. agarose or polyacrylamide and a buffer to be used in electrophoresis, HPLC columns, SSCP gels, formamide gels or a matrix support for MALDI-TOF).

It will be appreciated that the methods of the invention can be performed in conjunction with an analysis of other risk factors known to be associated with ACS. Such risk factors include epidemiological risk factors associated with an increased risk of developing ACS. Such risk factors include, but are not limited to smoking and/or exposure to tobacco smoke, age, sex and familial history. These risk factors can be used to augment an analysis of one or more polymorphisms as herein described when assessing a subject's risk of developing ACS.

It is recognized that individual SNPs may confer weak risk of susceptibility or protection to a disease or phenotype of interest. These modest effects from individual SNPs are typically measured as odds ratios in the order of 1-3. The specific phenotype of interest may be a disease, such as ACS, or an intermediate phenotype based on a pathological, biochemical or physiological abnormality (for example, impaired lung function). As described herein, when specific genotypes from individual SNPs are assigned a numerical value reflecting their phenotypic effect (for example, a positive value for susceptibility SNPs and a negative value for protective SNPs), the combined effects of these SNPs can be derived from an algorithm that calculates an overall score. Again as described herein in a case-control study design, this SNP score is linearly related to the frequency of disease (or likelihood of having disease).

The SNP score provides a means of comparing people with different scores and their odds of having disease in a simple dose-response relationship. In this analysis, the people with the lowest SNP score are the referent group (Odds ratio=1) and those with greater SNP scores have a correspondingly greater odds (or likelihood) of having the disease—again in a linear fashion. The Applicants believe, without wishing to be bound by any theory, that the extent to which combining SNPs optimises these analyses is dependent, at least in part, on the strength of the effect of each SNP individually in a univariate analysis (independent effect) and/or multivariate analysis (effect after adjustment for effects of other SNPs or non-genetic factors) and the frequency of the genotype from that SNP (how common the SNP is). However, the effect of combining certain SNPs may also be in part related to the effect that those SNPs have on certain pathophysiological pathways that underlie the phenotype or disease of interest.

When deriving a SNP score for each person, the score is the composite of any number of SNPs, with many SNPs making no contribution to the score—if the person does not carry the susceptibility or protective genetic variant for a specific SNP, the contribution of that SNP to the composite SNP score is 0. This is in sharp contrast to the multivariate analyses exemplified by the Framingham score (derived from the Framingham equations for heart disease which determine risk based on the combined effects of many parameters with each parameter conferring its own level of risk).

In addition to assigning risk to individuals based on their genetic SNP score, it is possible to segment a population when the frequency of the SNP score is compared between cases and controls and separation of the two distributions is achieved. The assignment of risk has utility in treating individuals (for example, prescribing a drug), whereas the segmentation of populations allows treatment strategies to be applied across populations (in for example a public health approach such as population-wide screening). Such treatment strategies may seek to optimise the application of one or more interventions amongst a population to achieve a given result, such as, for example, eradication of a communicable disease or to maximize cost-effectiveness. It should be noted that these separate utilities—the assignation of risk to an individual and the segmentation of a population—are independent of each other and the presence of the former does not predict the later (see, for example, Wald N J, et al., “When can a risk factor be used as a worthwhile screening test?” BMJ 1999; 319:1562-1565).

Therefore, in addition to utility in determining a subject's risk of developing ACS, a SNP score has clinical utility in helping to define a threshold or cut-off level in the SNP score that will define a subgroup of the population that is suitable to undergo an intervention. Such an intervention may be a diagnostic intervention, such as imaging test, other screening or diagnostic test (eg biochemical or RNA based test), or may be a therapeutic intervention, such as a chemopreventive or chemotherapeutic therapy, or a preventive lifestyle modification (such as stopping smoking). In defining this clinical threshold, people can be prioritized to a particular intervention in such a way to minimize costs or minimize risks of that intervention (for example, the costs of image-based screening or expensive preventive treatment or risk from drug side-effects or risk from radiation exposure). In determining this threshold, one might aim to maximize the ability of the test to detect the majority of cases (maximize sensitivity) but also to minimize the number of people at low risk that require, or may be are otherwise eligible for, the intervention of interest.

Receiver-operator curve (ROC) analyses analyze the clinical performance of a test by examining the relationship between sensitivity and false positive rate (i.e., 1-specificity) for a single variable in a given population. In an ROC analysis, the test variable may be derived from combining several factors. Either way, this type of analysis does not consider the frequency distribution of the test variable (for example, the SNP score) in the population and therefore the number of people who would need to be screened in order to identify the majority of those at risk but minimize the number who need to be screened or treated.

Determining a particular combination of SNPs to be used to generate a SNP score can enhance the ability to segment or subgroup people into intervention and non-intervention groups in order to better prioritize these interventions. Such an approach is useful in identifying which smokers might be best prioritized for interventions, such as screening for ACS. Such an approach could also be used for initiating treatments or other screening or diagnostic tests. As will be appreciated, this has important cost implications to offering such interventions.

Accordingly, the present invention also provides a method of assessing a subject's suitability for an intervention diagnostic of or therapeutic for ACS, the method comprising:

a) providing a net score for said subject, wherein the net score is or has been determined by:
i) providing the result of one or more genetic tests of a sample from the subject, and analyzing the result for the presence or absence of protective polymorphisms and for the presence or absence of susceptibility polymorphisms, wherein said protective and susceptibility polymorphisms are associated with ACS,
ii) assigning a positive score for each protective polymorphism and a negative score for each susceptibility polymorphism or vice versa;
iii) calculating a net score for said subject by representing the balance between the combined value of the protective polymorphisms and the combined value of the susceptibility polymorphisms present in the subject sample;
and

b) providing a distribution of net scores for ACS sufferers and non-sufferers wherein the net scores for ACS sufferers and non-sufferers are or have been determined in the same manner as the net score determined for said subject;

c) determining whether the net score for said subject lies within a threshold on said distribution separating individuals deemed suitable for said intervention from those for whom said intervention is deemed unsuitable;

wherein a net score within said threshold is indicative of the subject's suitability for the intervention, and wherein a net score outside the threshold is indicative of the subject's unsuitability for the intervention.

The value assigned to each protective polymorphism may be the same or may be different. The value assigned to each susceptibility polymorphism may be the same or may be different, with either each protective polymorphism having a negative value and each susceptibility polymorphism having a positive value, or vice versa.

The intervention may be a diagnostic test for the disease, such as a blood test or a CT scan for ACS. Alternatively, the intervention may be a therapy for the disease, such as chemotherapy or radiotherapy, including a preventative therapy for the disease, such as the provision of motivation to the subject to stop smoking.

A distribution of SNP scores for ACS sufferers and resistant smoker controls (non-sufferers) can be established using the methods of the invention. For example, a distribution of SNP scores derived from a 7 SNP panel consisting of the protective and susceptibility polymorphisms Y402H C/T in the gene encoding Complement Factor H (CFH), Asp92Asn A/G in the gene encoding Myeloid IgA Fc receptor (FCAR), A/G (rs4804611) in the gene encoding Zinc finger protein 627 (ZNF627), Asn159Asn A/G in the gene encoding Serpin 2, C3279T A/G in the gene encoding Galectin-2 (LGALS2), A387P C/G in the gene encoding Thrombospondin 4, and Asp51Ala A/C in the gene encoding Interleukin 1 family, member 10 (ILIF10), among ACS sufferers and non-sufferers is determined. A threshold SNP score can be determined that separates people into intervention and non-intervention groups, so as to better prioritize those individuals suitable for such interventions.

The implementation of such methods in computer systems and programs as described herein, the data produced by such methods, and the use of such data in the determination of a subject's suitability or unsuitability for an intervention diagnostic or therapeutic of ACS, of arterial inflammation, or of ACS-associated impaired vascular function, are also contemplated.

As used herein, the phrase “assessing a subject's suitability for an intervention” or grammatical equivalents thereof means one or more determinations of whether a given subject is or should be a candidate for an intervention or is not or should not be a candidate for an intervention. Preferably, the assessment involves a determination of the subject's SNP score in relation to a distribution of SNP scores as described herein.

As used herein the term “intervention” includes medical tests, analyses, and treatments, including diagnostic, therapeutic and preventative treatments, and psychological or psychiatric tests, analyses and treatments, including counseling and the like.

Computer-Related Embodiments

It will also be appreciated that the methods of the invention are amenable to use with and the results analyzed by computer systems, software and processes. Computer systems, software and processes to identify and analyze genetic polymorphisms are well known in the art. Similarly, implementation of the algorithm utilized to generate a SNP score as described herein in computer systems, software and processes is also contemplated. For example, the results of one or more genetic analyses as described herein may be analyzed using a computer system and processed by such a system utilizing a computer-executable example of the algorithm described herein.

Both the SNPs and the results of an analysis of the SNPs utilized in the present invention may be “provided” in a variety of mediums to facilitate use thereof. As used in this section, “provided” refers to a manufacture, other than an isolated nucleic acid molecule, that contains SNP information of the present invention. Such a manufacture provides the SNP information in a form that allows a skilled artisan to examine the manufacture using means not directly applicable to examining the SNPs or a subset thereof as they exist in nature or in purified form. The SNP information that may be provided in such a form includes any of the SNP information provided by the present invention such as, for example, polymorphic nucleic acid and/or amino acid sequence information, information about observed SNP alleles, alternative codons, populations, allele frequencies, SNP types, and/or affected proteins, identification as a protective SNP or a susceptibility SNP, weightings (for example for use in an algorithm utilized to derive a SNP score as described herein), or any other information provided by the present invention in Tables 1-9 and/or the Sequence ID Listing.

In one application of this embodiment, the SNPs and the results of an analysis of the SNPs utilized in the present invention can be recorded on a computer readable medium. As used herein, “computer readable medium” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon SNP information of the present invention. One such medium is provided with the present application, namely, the present application contains computer readable medium (floppy disc) that has nucleic acid sequences used in analyzing the SNPs utilized in the present invention provided/recorded thereon in ASCII text format in a Sequence Listing along with accompanying Tables that contain detailed SNP and sequence information.

As used herein, “recorded” refers to a process for storing information on computer readable medium. A skilled artisan can readily adopt any of the presently known methods for recording information on computer readable medium to generate manufactures comprising the SNP information of the present invention.

A variety of data storage structures are available to a skilled artisan for creating a computer readable medium having recorded thereon SNP information of the present invention. The choice of the data storage structure will generally be based on the means chosen to access the stored information. In addition, a variety of data processor programs and formats can be used to store the SNP information of the present invention on computer readable medium. For example, sequence information can be represented in a word processing text file, formatted in commercially-available software such as WordPerfect and Microsoft Word, represented in the form of an ASCII file, or stored in a database application, such as OB2, Sybase, Oracle, or the like. A skilled artisan can readily adapt any number of data processor structuring formats (e.g., text file or database) in order to obtain computer readable medium having recorded thereon the SNP information of the present invention.

By providing the SNPs and/or the results of an analysis of the SNPs utilized in the present invention in computer readable form, a skilled artisan can routinely access the SNP information for a variety of purposes. Computer software is publicly available which allows a skilled artisan to access sequence information provided in a computer readable medium. Examples of publicly available computer software include BLAST (Altschul et at, J. Mol. Biol. 215:403-410 (1990)) and BLAZE (Brutlag et at, Comp. Chem. 17:203-207 (1993)) search algorithms.

The present invention further provides systems, particularly computer-based systems, which contain the SNP information described herein. Such systems may be designed to store and/or analyze information on, for example, a number of SNP positions, or information on SNP genotypes from a number of individuals. The SNP information of the present invention represents a valuable information source. The SNP information of the present invention stored/analyzed in a computer-based system may be used for such applications as identifying subjects at risk of ACS, in addition to computer-intensive applications as determining or analyzing SNP allele frequencies in a population, mapping disease genes, genotype-phenotype association studies, grouping SNPs into haplotypes, correlating SNP haplotypes with response to particular drugs, or for various other bioinformatic, pharmacogenomic, drug development, or human identification/forensic applications.

As used herein, “a computer-based system” refers to the hardware, software, and data storage used to analyze the SNP information of the present invention. The minimum hardware of the computer-based systems of the present invention typically comprises a central processing unit (CPU), an input, an output, and data storage. A skilled artisan can readily appreciate that any one of the currently available computer-based systems are suitable for use in the present invention. Such a system can be changed into a system of the present invention by utilizing the SNP information, such as that provided herewith on the floppy disc, or a subset thereof, without any experimentation.

As stated above, the computer-based systems of the present invention comprise data storage having stored therein SNP information, such as SNPs and/or the results of an analysis of the SNPs utilized in the present invention, and the necessary hardware and software for supporting and implementing one or more programs or algorithms. As used herein, “data storage” refers to memory which can store SNP information of the present invention, or a memory access facility which can access manufactures having recorded thereon the SNP information of the present invention.

The one or more programs or algorithms are implemented on the computer-based system to identify or analyze the SNP information stored within the data storage. For example, such programs or algorithms can be used to determine which nucleotide is present at a particular SNP position in a target sequence, to analyze the results of a genetic analysis of the SNPs described herein, or to derive a SNP score as described herein. As used herein, a “target sequence” can be any DNA sequence containing the SNP position(s) to be analyzed, searched or queried.

A variety of structural formats for the input and output can be used to input and output the information in the computer-based systems of the present invention. An exemplary format for an output is a display that depicts the SNP information, such as the presence or absence of specified nucleotides (alleles) at particular SNP positions of interest, or the derived SNP score for a subject. Such presentation can provide a rapid, binary scoring system for many SNPs or subjects simultaneously. It will be appreciated that such output may be accessed remotely, for example over a LAN or the internet. Typically, given the nature of SNP information, such remote accessing of such output or of the computer system itself is available only to verified users so that the security of the SNP information and/or the computer system is maintained. Methods to control access to computer systems and the data residing thereon are well-known in the art, and are amenable to the embodiments of the present invention.

One exemplary embodiment of a computer-based system comprising SNP information of the present invention that can be used to implement the present invention includes a processor connected to a bus. Also connected to the bus are a main memory (preferably implemented as random access memory, RAM) and a variety of secondary storage devices, such as a hard drive and a removable medium storage device. The removable medium storage device may represent, for example, a floppy disc drive, a CD-ROM drive, a magnetic tape drive, etc. A removable storage medium (such as a floppy disc, a compact disc, a magnetic tape, etc.) containing control logic and/or data recorded therein may be inserted into the removable medium storage device. The computer system includes appropriate software for reading the control logic and/or the data from the removable storage medium once inserted in the removable medium storage device. The SNP information of the present invention may be stored in a well-known manner in the main memory, any of the secondary storage devices, and/or a removable storage medium. Software for accessing and processing the SNP information (such as SNP scoring tools, search tools, comparing tools, etc.) preferably resides in main memory during execution. Accordingly, the present invention provides a system for determining a subject's risk of developing ACS, said system comprising:

computer processor means for receiving, processing and communicating data;

storage means for storing data including a reference genetic database of the results of at least one genetic analysis with respect to ACS and optionally a reference non-genetic database of non-genetic risk factors for ACS; and

a computer program embedded within the computer processor which, once data consisting of or including the result of a genetic analysis for which data is included in the reference genetic database is received, processes said data in the context of said reference databases to determine, as an outcome, the subject's risk of developing ACS, said outcome being communicable once known, preferably to a user having input said data.

Preferably, the at least one genetic analysis is an analysis of one or more polymorphisms selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2;
C3279T A/G in the gene encoding Galectin-2 (LGALS2); or
one or more polymorphisms which are in linkage disequilibrium with said one or more polymorphisms.

In one embodiment, the data is input by a representative of a healthcare provider.

In another embodiment, the data is input by the subject, their medical advisor or other representative.

Preferably, said system is accessible via the internet or by personal computer.

Preferably, said reference genetic database consists of, comprises or includes the results of an ACS-associated genetic analysis selected from one or more of the genetic analyses described herein and/or the Cardiogene™-brand cardiovascular test, preferably the results of an analysis of one or more polymorphisms selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2;
C3279T A/G in the gene encoding Galectin-2 (LGALS2); or
one or more polymorphisms which are in linkage disequilibrium with said one or more polymorphisms.

More preferably, said reference genetic database consists of, comprises or includes the results of an analysis of any two, any three, any four, or all of the polymorphisms selected from the group consisting of:
Y402H C/T in the gene encoding Complement Factor H (CFH);
Asp92Asn A/G in the gene encoding Myeloid IgA Fc receptor (FCAR);
A/G (rs4804611) in the gene encoding Zinc finger protein 627 (ZNF627);
Asn159Asn A/G in the gene encoding Serpin 2; or
C3279T A/G in the gene encoding Galectin-2 (LGALS2).

The reference genetic database may additionally comprise or include the results of an analysis of one or more further polymorphisms selected from the group consisting of:
A387P C/G in the gene encoding Thrombospondin 4; or
Asp51Ala A/C in the gene encoding Interleukin 1 family, member 10 (ILIF10).

More preferably, said reference genetic database consists of, comprises or includes the results of all of the genetic analyses described herein and the Cardiogene™-brand cardiovascular test.

The present invention further provides a computer program for use in a computer system as described, and the use of the results of such systems and programs in the determination of a subject's risk of developing ACS, or in determining the suitability of a subject for an intervention as described herein.

As used herein, the Cardiogene™-brand cardiovascular test comprises the methods of determining a subject's predisposition to and/or potential risk of developing acute coronary syndrome (ACS) and related methods as defined in New Zealand Patent Application No. 543520, filed Nov. 10, 2005; New Zealand Patent Application No. 543985, filed Dec. 6, 2005; New Zealand Patent Application No. 549951, filed Sep. 15, 2006; and PCT International Application PCT/NZ2006/000292, filed Nov. 10, 2006 (published as WO2007/055602), each of the foregoing which is incorporated herein by reference in its entirety.

In particular, the Cardiogene™-brand cardiovascular test includes a method of determining a subject's risk of developing ACS comprising analyzing a sample from said subject for the presence or absence of one or more polymorphisms selected from the group consisting of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2); or
372 T/C in the gene encoding Tissue inhibitor of metalloproteinase 1 (TIMP1);

wherein the presence or absence of one or more of said polymorphisms is indicative of the subject's risk of developing ACS.

The method of the Cardiogene™-brand cardiovascular test can additionally comprise analyzing a sample from said subject for the presence of one or more further polymorphisms selected from the group consisting of:
−509 C/T in the gene encoding Transforming growth factor β1 (TGFB1);
Thr26Asn A/C in the gene encoding Lymphotoxin α (LTA);
Asp299Gly A/G in the gene encoding Toll-like Receptor 4 (TLR4);

Thr399Ile C/T in the gene encoding TLR4;
−63 T/A in the gene encoding Nuclear factor of kappa light polypeptide gene enhancer in B-cells inhibitor-like 1 (NFKBIL1);
−1630 Ins/Del (AACTT/Del) in the gene encoding Platelet derived growth factor receptor alpha (PDGFRA);
−1607 1G/2G (Del/G) in the gene encoding Matrix metalloproteinase 1 (MMP1);
12 IN 5 C/T in the gene encoding Platelet derived growth factor alpha (PDGFA);
−588 C/T in the gene encoding Glutamate-cysteine ligase modifier subunit (GCLM);

Ile132Val A/G in the gene encoding Olfactory receptor analogue OR13G1 (OR13G1);
Glu288Val A/T (M/S) in the gene encoding alpha 1-antitrypsin (α1-AT);
K469E A/G in the gene encoding Intracellular adhesion molecule 1 (ICAM1);
−23 C/G in the gene encoding HLA-B associated transcript 1 (BAT1);
Glu298Asp G/T in the gene encoding Nitric Oxide synthase 3 (NOS3);
−668 4G/5G in the gene encoding Plasminogen activator inhibitor 1 (PAI-1); or
−181 A/G in the gene encoding Matrix metalloproteinase 7 (MMP7).

As in the methods described herein, in the Cardiogene™-brand cardiovascular test the one or more polymorphisms can be detected directly or by detection of one or more polymorphisms which are in linkage disequilibrium with said one or more polymorphisms.

The predictive methods of the invention allow a number of therapeutic interventions and/or treatment regimens to be assessed for suitability and implemented for a given subject. The simplest of these can be the provision to the subject of motivation to implement a lifestyle change, for example, where the subject is a current smoker, the methods of the invention can provide motivation to quit smoking.

The manner of therapeutic intervention or treatment will be predicated by the nature of the polymorphism(s) and the biological effect of said polymorphism(s). For example, where a susceptibility polymorphism is associated with a change in the expression of a gene, intervention or treatment is preferably directed to the restoration of normal expression of said gene, by, for example, administration of an agent capable of modulating the expression of said gene. Where a polymorphism is associated with decreased expression of a gene, therapy can involve administration of an agent capable of increasing the expression of said gene, and conversely, where a polymorphism is associated with increased expression of a gene, therapy can involve administration of an agent capable of decreasing the expression of said gene. Methods useful for the modulation of gene expression are well known in the art. For example, in situations where a polymorphism is associated with upregulated expression of a gene, therapy utilizing, for example, RNAi or antisense methodologies can be implemented to decrease the abundance of mRNA and so decrease the expression of said gene. Alternatively, therapy can involve methods directed to, for example, modulating the activity of the product of said gene, thereby compensating for the abnormal expression of said gene.

Where a susceptibility polymorphism is associated with decreased gene product function or decreased levels of expression of a gene product, therapeutic intervention or treatment can involve augmenting or replacing of said function, or supplementing the amount of gene product within the subject for example, by administration of said gene product or a functional analogue thereof. For example, where a polymorphism is associated with decreased enzyme function, therapy can involve administration of active enzyme or an enzyme analogue to the subject. Similarly, where a polymorphism is associated with increased gene product function, therapeutic intervention or treatment can involve reduction of said function, for example, by administration of an inhibitor of said gene product or an agent capable of decreasing the level of said gene product in the subject. For example, where a SNP allele or genotype is associated with increased enzyme function, therapy can involve administration of an enzyme inhibitor to the subject.

Likewise, when a protective polymorphism is associated with upregulation of a particular gene or expression of an enzyme or other protein, therapies can be directed to mimic such upregulation or expression in an individual lacking the resistive genotype, and/or delivery of such enzyme or other protein to such individual Further, when a protective polymorphism is associated with downregulation of a particular gene, or with diminished or eliminated expression of an enzyme or other protein, desirable therapies can be directed to mimicking such conditions in an individual that lacks the protective genotype.

The relationship between the various polymorphisms identified above and the susceptibility (or otherwise) of a subject to ACS also has application in the design and/or screening of candidate therapeutics. This is particularly the case where the association between a polymorphism predictive of susceptibility is manifested by either an upregulation or downregulation of expression of a gene. In such instances, the effect of a candidate therapeutic on such upregulation or downregulation is readily detectable.

For example, in one embodiment existing human vascular organ and cell cultures are screened for SNP genotypes as set forth above. (For information on human vascular organ and cell cultures, see for example: Clare Wise ED., Epithelial Cell Culture Protocols, 2002, ISBN 0896038939, Humana Press Inc. NJ; Endothelial Cell Culture, Roy Bicknell, ED., 1996, ISBN 0521550246, Cambridge University Press, UK; Cell Culture Models of Biological Barriers, Claus-Michael Lehr, ED., 2002, ISBN 0415277248, Taylor and Francis, UK; each of which is hereby incorporated by reference in its entirety.) Cultures representing relevant genotype groups are selected, together with cultures which are putatively “normal” in terms of the expression of a gene which is either upregulated or downregulated where a polymorphism is present.

Samples of such cultures are exposed to a library of candidate therapeutic compounds and screened for: (a) downregulation of genes that are normally upregulated in susceptibility genotypes; or (b) upregulation of genes that are normally downregulated in susceptibility genotypes. Compounds are selected for their ability to alter the regulation and/or action of genes in a culture having a susceptibility genotype.

Similarly, where the polymorphism is one which when present results in a physiologically active concentration of an expressed gene product outside of the normal range for a subject (adjusted for age and sex), and where there is an available prophylactic or therapeutic approach to restoring levels of that expressed gene product to within the normal range, individual subjects can be screened to determine the likelihood of their benefiting from that restorative approach. Such screening involves detecting the presence or absence of the polymorphism in the subject by any of the methods described herein, with those subjects in which the polymorphism is present being identified as individuals likely to benefit from treatment.

The invention will now be described in more detail, with reference to the following non-limiting examples.

### Example 1

### Case Association Study

### Introduction

Case-control association studies allow the careful selection of a control group where matching for important risk factors is critical. In this study, smokers diagnosed with ACS and smokers without ACS were compared. This unique control group is highly relevant as it is impossible to pre-select smokers with zero risk of ACS—i.e., those who although smokers will never develop ACS. Smokers with a high pack year history and no known cardiovascular disease were used as a “low risk” group of smokers, as the Applicants believe it is not possible with current knowledge to identify a lower risk group of smokers. The Applicants believe, without wishing to be bound by any theory, that this approach allows for a more rigorous comparison of low penetrant, high frequency polymorphisms that may confer an increased risk of developing ACS. The Applicants also believe, again without wishing to be bound by any theory, that there may be polymorphisms that confer a degree of protection from ACS which may only be evident if a smoking cohort with normal cardiovascular function is utilized as a comparator group. Thus, smokers with ACS would be expected to have a lower frequency of these polymorphisms compared to smokers with normal cardiovascular function and no diagnosed ACS.

Subjects of European decent who had smoked a minimum of fifteen pack years and diagnosed with acute coronary syndrome (ACS, including acute myocardial infarction and unstable angina) were recruited. Subjects met the following criteria: diagnosed with ACS based on clinical presentation (history, ECG, cardiac biomarker assays) to a tertiary care hospital. Subjects with ACS had had coronary angiograms that confirmed the presence of atheromatous disease of the coronary arteries. Subjects with ACS were aged between 40-60 yrs old and of European descent. One hundred and forty-eight subjects were recruited, of these 85% were male, the mean FEV1/FVC (±1SD) was 74% (±8), mean FEV1 as a percentage of predicted was 94 (±15). Mean age, cigarettes per day and pack year history was 50 yrs (±3), 22 cigarettes/day (±8) and 31 pack years (±11), respectively. Four hundred and sixty European subjects who had smoked a minimum of fifteen pack years and who had never suffered from angina, chest pain, suffered a heart attack, or had been diagnosed with ischaemic heart disease in the past were also studied. This control group was recruited through community based volunteers who were ex-smokers or current smokers, and consisted 55% male, with a mean FEV1/FVC (±1 SD) of 75% (±9), and mean FEV1 as a percentage of predicted was 98 (±12). Mean age, cigarettes per day and pack year history was 60 yrs (±10), 23 cigarettes/day (±11) and 40 pack years (±21), respectively.

This study shows that polymorphisms found in greater frequency in acute coronary syndrome patients compared to resistant smokers may reflect an increased susceptibility to the development of life-threatening acute coronary syndrome. Similarly, polymorphisms found in greater frequency in resistant smokers compared to acute coronary syndrome patients may reflect a protective role.

TABLE 1
Summary of characteristics for the ACS
cohort and resistant control smokers.

| | Acute Coronary | | |
|---|---|---|---|
| Parameter | syndrome | Resistant smokers | |
| Mean (1SD) | N = 148 | N = 460 | Differences |
| % male | 85% | 55% | P < 0.05 |
| Age (yrs) | 50 (3) | 60 (10) | P < 0.05 |
| Pack years | 31 (11) | 40 (21) | P < 0.05 |
| Cigarettes/day | 22 (8) | 23 (11) | ns |
| FEV1 (L) | 3.3 (0.7) | 2.7 (0.6) | P < 0.05 |
| FEV1 % predict | 94 (15) | 98% (12) | P < 0.05 |
| FEV1/FVC | 74 (8) | 75 (9) | P < 0.05 |

Means and 1SD

### Genotyping Methods

### Polymorphism Genotyping Using the Sequenom Autoflex Mass Spectrometer

Genomic DNA was extracted from whole blood samples (Maniatis, T., Fritsch, E. F. and Sambrook, J., Molecular Cloning Manual. 1989). Purified genomic DNA was aliquoted (10 ng/ul concentration) into 96 well plates and genotyped on a Sequenom™ system (Sequenom™ Autoflex Mass Spectrometer and Samsung 24 pin nanodispenser) using the following sequences, amplification conditions and methods.

The following conditions were used for the PCR multiplex reaction: final concentrations were for 10× Buffer 15 mM MgCl₂ 1.25×, 25 mM MgCl₂ 1.625 mM, dNTP mix 25 mM 500 uM, primers 4 uM 100 nM, Taq polymerase (Quiagen hot start) 0.15 U/reaction, Genomic DNA 10 ng/ul. Cycling times were 95° C. for 15 min, (5° C. for 15 s, 56° C. 30 s, 72° C. 30 s for 45 cycles with a prolonged extension time of 3 min to finish. We used shrimp alkaline phosphatase (SAP) treatment (2 ul to 5 ul per PCR reaction) incubated at 35° C. for 30 min and extension reaction (add 2 ul to 7 ul after SAP treatment) with the following volumes per reaction of: water, 0.76 ul; hME 10× termination buffer, 0.2 ul; hME primer (10 uM), 1 ul; Mass EXTEND enzyme, 0.04 ul. See Tables 1-10 for full name of SNPs and candidate genes.

TABLE 2.1
Sequenom conditions for PCR and Mass spectrometer genotyping

| | | | |
|---|---|---|---|
| SNP | SNP_ID | 2nd-PCRP | 1st-PCRP |

| | | | |
|---|---|---|---|
| CFH | rs1061170 | ACGTTGGATGGTTATGGTCCTTAGGAAAATG | ACGTTGGATGGGCAACGTCTATAGATTTACC |
| | | [SEQ.ID.NO. 1] | [SEQ.ID.NO. 2] |
| FCAR | rs11666735 | ACGTTGGATGGACCCTGGATGTTTCCTTAC | ACGTTGGATGGCCAATATAGGATAGGGCAC |
| | | [SEQ.ID.NO. 3] | [SEQ.ID.NO. 4] |
| THSP4 | rs1866389 | ACGTTGGATGTTAACGCAGATCGAGTTGGG | ACGTTGGATGTTTCTGCACTAGGTCTGCAC |
| | | [SEQ.ID.NO. 5] | [SEQ.ID.NO. 6] |
| ZNF627 | rs4804611 | ACGTTGGATGGCCAATTATCTTACAGGGTC | ACGTTGGATGTTGGGAAAGCCTTCAGTCCT |
| | | [SEQ.ID.NO. 7] | [SEQ.ID.NO. 8] |
| ILIF10 | rs6743376 | ACGTTGGATGTCCCTCCTAGAGAAGATCTG | ACGTTGGATGCCTGGATCCCCAGGAAAATG |
| | | [SEQ.ID.NO. 9] | [SEQ.ID.NO. 10] |
| Serpin2 | rs6747096 | ACGTTGGATGGGAGTCTAACTCATGCTTC | ACGTTGGATGTGATTCCATCAATGCATGGG |
| | | [SEQ.ID.NO. 11] | [SEQ.ID.NO. 12] |
| LGALS2 | rs7291467 | ACGTTGGATGGAGCCATCTCCTGATGCTTG | ACGTTGGATGCACACAGACACTCACAGACG |
| | | [SEQ.ID.NO. 13] | [SEQ.ID.NO. 14] |

TABLE 2.2
Sequenom conditions for PCR and Mass spectrometer genotyping

| SNP | SNP_ID | AMP_LEN | UP_CONF | MP_CONF | Tm (NN) | PcGC | PWARN |
|---|---|---|---|---|---|---|---|
| CFH | rs1061170 | 120 | 83.8 | 61.5 | 46.2 | 20 | |
| FCAR | rs11666735 | 103 | 100 | 89.7 | 52.9 | 58.8 | d |
| THSP4 | rs1866389 | 94 | 99.9 | 61.5 | 53.9 | 64.7 | d |
| ZNF627 | rs4804611 | 103 | 96 | 61.5 | 48.3 | 50 | |
| ILIF10 | rs6743376 | 99 | 98.6 | 61.5 | 46.1 | 56.3 | D |
| Serpin2 | rs6747096 | 120 | 94.9 | 61.5 | 48.4 | 56.3 | D |
| LGALS2 | rs7291467 | 89 | 96.7 | 61.5 | 45.5 | 41.2 | D |

TABLE 2.3
Sequenom conditions for PCR and Mass spectrometer genotyping

| | | | | | | | |
|---|---|---|---|---|---|---|---|
| | | UEP_{—} | UEP_{—} | | EXT1_{—} | EXT1_{—} | |
| SNP | SNP_ID | DIR | MASS | UEP_SEQ | CALL | MASS | EXT1_SEQ |

| | | | | | | | |
|---|---|---|---|---|---|---|---|
| CFH | rs1061170 | F | 7736.1 | TTTGGAAAATGGATATAATCAAAAT | C | 7983.3 | TTTGGAAAATGGATATAATCAAAATC |
| | | | | [SEQ.ID.NO. 15] | | | [SEQ.ID.NO. 16] |
| FCAR | rs11666735 | R | 5186.4 | TACCAGCTCCAGGGTGT | G | 5433.6 | TACCAGCTCCAGGGTGTC |
| | | | | [SEQ.ID.NO. 17] | | | [SEQ.ID.NO. 18] |
| THSP4 | rs1866389 | F | 6561.3 | ggagCGAGTTGGGAACGCACG | C | 6808.4 | ggagCGAGTTGGGAACGCACGC |
| | | | | [SEQ.ID.NO. 19] | | | [SEQ.ID.NO. 20] |
| ZNF627 | rs4804611 | R | 5425.5 | TACAGGGTCTTTCTCCAC | G | 5672.7 | TACAGGGTCTTTCTCCACC |
| | | | | [SEQ.ID.NO. 21] | | | [SEQ.ID.NO. 22] |
| ILIF10 | rs6743376 | F | 5235.4 | gCCTAACAGAGGCTTGG | C | 5482.6 | gCCTAACAGAGGCTTGGC |
| | | | | [SEQ.ID.NO. 23] | | | [SEQ.ID.NO. 24] |
| Serpin2 | rs6747096 | F | 5996.9 | aacaACTCACCCCTGGTTTC | A | 6268.1 | aacaACTCACCCCTGGTTTCA |
| | | | | [SEQ.ID.NO. 25] | | | [SEQ.ID.NO. 26] |
| LGALS2 | rs7291467 | R | 5584.6 | aTGATGCTTGGTGTTAGA | G | 5831.8 | aTGATGCTTGGTGTTAGAC |
| | | | | [SEQ.ID.NO. 27] | | | [SEQ.ID.NO. 28] |

TABLE 2.4
Sequenom conditions for PCR and Mass spectrometer genotyping

| | | | | |
|---|---|---|---|---|
| SNP | SNP_ID | EXT2_CALL | EXT2_MASS | EXT2_SEQ |

| | | | | |
|---|---|---|---|---|
| CFH | rs1061170 | T | 8063.2 | TTTGGAAAATGGATATAATCAAAATT |
| | | | | [SEQ.ID.NO. 29] |
| FCAR | rs11666735 | A | 5513.5 | TACCAGCTCCAGGGTGTT |
| | | | | [SEQ.ID.NO. 30] |
| THSP4 | rs1866389 | G | 6848.5 | ggagCGAGTTGGGAACGCACGG |
| | | | | [SEQ.ID.NO. 31] |
| ZNF627 | rs4804611 | A | 5752.6 | TACAGGGTCTTTCTCCACT |
| | | | | [SEQ.ID.NO. 32] |
| ILIF10 | rs6743376 | A | 5506.6 | gCCTAACAGAGGCTTGGA |
| | | | | [SEQ.ID.NO. 33] |
| Serpin2 | rs6747096 | G | 6284.1 | aacaACTCACCCCTGGTTTCG |
| | | | | [SEQ.ID.NO. 34] |
| LGALS2 | rs7291467 | A | 5911.7 | aTGATGCTTGGTGTTAGAT |
| | | | | [SEQ.ID.NO. 35] |

### Results

TABLE 3
Complement Factor H Y402H C/T polymorphism allele
and genotype frequencies in the ACS patients and
resistant smokers.

| | Allele* | | Genotype | | |
|---|---|---|---|---|---|
| Frequency | C | T | CC | CT | TT |
| ACS n = 148 | 102 | 194 | 21 | 60 | 67 |
| (%) | (34%) | (66%) | (14%) | (41%) | (45%) |
| Resistant n = 456 | 354 | 558 | 62 | 230 | 164 |
| (%) | (39%) | (61%) | (14%) | (50%) | (36%) |

number of chromosomes (2n)
Genotype. TT vs CT/CC for ACS vs resistant smoker controls, Odds ratio (OR) = 1.5, 95% confidence limits = 1.0-2.2, χ² (Mantel-Haenszel) = 4.09, p = 0.04, TT genotype = susceptibility

TABLE 4
Myeloid IgA Fc receptor (FCAR) Asp92Asn A/G
polymorphism allele and genotype frequencies in the ACS
patients and resistant smokers.

| | Allele* | | Genotype | | |
|---|---|---|---|---|---|
| Frequency | A | G | AA | AG | GG |
| ACS n = 149 | 22 | 276 | 5 | 12 | 132 |
| (%) | (7%) | (93%) | (3%) | (8%) | (89%) |
| Resistant n = 461 | 73 | 849 | 3 | 67 | 391 |
| (%) | (8%) | (92%) | (1%) | (15%) | (85%) |

number of chromosomes (2n)
Genotype. AA/AG vs GG for ACS vs resistant smoker controls, Odds ratio (OR) = 0.63, 95% confidence limits = 0.34-1.2, χ² (Mantel-Haenszel) = 2.30, p = 0.13, AA/AG genotype = protective (GG susceptibility)

TABLE 5
Thrombospondin 4 A387P C/G polymorphism allele and
genotype frequencies in the ACS patients and resistant smokers.

| | Allele* | | Genotype | | |
|---|---|---|---|---|---|
| Frequency | C | G | CC | CG | GG |
| ACS n = 146 | 235 | 57 | 93 | 49 | 4 |
| (%) | (80%) | (20%) | (64%) | (33%) | (3%) |
| Resistant n = 457 | 683 | 231 | 259 | 165 | 33 |
| (%) | (75%) | (25%) | (57%) | (36%) | (7%) |

number of chromosomes (2n)
Genotype. GG vs CC/CG for ACS vs resistant smoker controls, Odds ratio (OR) = 0.36, 95% confidence limits = 0.11-11, χ² (Mantel-Haenszel) = 3.82, p = 0.05, GG genotype = protective
Allele G vs C, ACS vs resistant smoker controls, Odds ratio (OR) = 0.72, 95% confidence limits = 0.51-1.0, χ² (Mantel-Haenszel) = 4.03, p = 0.04, G allele = protective

TABLE 6
Zinc finger protein (ZNF) 627 A/G (rs4804611) polymorphism allele
and genotype frequencies in the ACS patients and resistant smokers.

| | |
|---|---|
| Allele* | Genotype |

| Frequency | A | G | AA | AG | GG |
|---|---|---|---|---|---|
| ACS n = 144 | 193 | 95 | 66 | 61 | 17 |
| (%) | (67%) | (33%) | (46%) | (42%) | (12%) |
| Resistant n = 436 | 655 | 217 | 253 | 149 | 34 |
| (%) | (75%) | (25%) | (58%) | (34%) | (8%) |

number of chromosomes (2n)
Genotype. GA/GG vs AA for ACS vs resistant smoker controls, Odds ratio (OR) = 1.63, 95% confidence limits = 1.1-2.43, χ² (Mantel-Haenszel) = 6.49, p = 0.01, GA/GG genotype = susceptibility (AA protective)
Allele G vs A, ACS vs resistant smoker controls, Odds ratio (OR) = 1.49, 95% confidence limits = 1.1-2.0, χ² (Mantel-Haenszel) = 7.22, p = 0.07, G allele = susceptibility

TABLE 7
Interleukin 1 family, member 10 (ILIF10) Asp51Ala A/C
polymorphism allele and genotype frequencies in the ACS
patients and resistant smokers.

| | Allele* | | Genotype | | |
|---|---|---|---|---|---|
| Frequency | A | C | AA | AC | CC |
| ACS n = 147 | 172 | 122 | 56 | 60 | 31 |
| (%) | (59%) | (41%) | (38%) | (41%) | (21%) |
| Resistant n = 452 | 577 | 327 | 176 | 225 | 51 |
| (%) | (64%) | (36%) | (39%) | (50%) | (11%) |

number of chromosomes (2n)
Genotype. CC vs AA/AC for ACS vs resistant smoker controls, Odds ratio (OR) = 2.10, 95% confidence limits = 1.3-3.5, χ² (Mantel-Haenszel) = 9.01, p = 0.003, CC genotype = susceptibility
Allele C vs A, ACS vs resistant smoker controls, Odds ratio (OR) = 1.25, 95% confidence limits = 0.95-1.65, χ² (Mantel-Haenszel) = 2.68, p = 0.10, C allele = susceptibility

TABLE 8
Serpin 2 Asn159Asn A/G polymorphism allele and genotype
frequencies in the ACS patients and resistant smokers.

| | Allele* | | Genotype | | |
|---|---|---|---|---|---|
| Frequency | A | G | AA | AG | GG |
| ACS n = 147 | 231 | 63 | 87 | 57 | 3 |
| (%) | (79%) | (21%) | (59%) | (39%) | (2%) |
| Resistant n = 453 | 739 | 167 | 300 | 139 | 14 |
| (%) | (82%) | (18%) | (66%) | (31%) | (3%) |

number of chromosomes (2n)
Genotype. AG/GG vs GG for ACS vs resistant smoker controls, Odds ratio (OR) = 1.35, 95% confidence limits = 0.9-2.0, χ² (Mantel-Haenszel) = 2.41, p = 0.12, AG/GG genotype = susceptibility (AA protective)

TABLE 9
Galectin-2 (LGALS2) C3279T A/G polymorphism allele and
genotype frequencies in the ACS patients and resistant smokers.

| | Allele* | | Genotype | | |
|---|---|---|---|---|---|
| Frequency | A | G | AA | AG | GG |
| ACS n = 147 | 190 | 104 | 60 | 70 | 17 |
| (%) | (65%) | (35%) | (41%) | (48%) | (12%) |
| Resistant n = 451 | 530 | 372 | 155 | 220 | 76 |
| (%) | (59%) | (41%) | (34%) | (49%) | (17%) |

number of chromosomes (2n)
Genotype. GG vs AA/AG for ACS vs resistant smoker controls, Odds ratio (OR) = 0.65, 95% confidence limits = 0.4-1.2, χ² (Mantel-Haenszel) = 2.36, p = 0.12, GG genotype = protective
Allele G vs A, ACS vs resistant smoker controls, Odds ratio (OR) = 0.78, 95% confidence limits = 0.59-1.0, χ² (Mantel-Haenszel) = 3.18, p = 0.07, G allele = protective

Table 10 below presents a summary of the protective and susceptibility SNPs identified herein.

TABLE 10
Summary of Protective and susceptibility SNPs for ACS

| | | | | | | |
|---|---|---|---|---|---|---|
| Gene | Polymorphism | Rs# | Genotype | Phenotype | OR | P value |

| | | | | | | |
|---|---|---|---|---|---|---|
| CFH | Y402 H | 1061170 | TT | susceptibility | 1.5 | 0.04 |
| FCAR (IgA Fc receptor) | Asp92Asn | 11666735 | AA/AG | protective | 0.63 | 0.13 |
| | | | GG | (susceptibility) | | |
| Thrombospondin 4 | A387P | 1866389 | GG | protective | 0.36 | 0.05 |
| ZNF627 | A/G | 4804611 | GA/GG | susceptibility | 1.42 | 0.07 |
| | | | AA | (protective) | | |
| IL1F10 | Asp51Ala | 6743376 | CC | susceptibility | 2.10 | 0.003 |
| Serpin 2 | Asn159Asn | 6747096 | AG/GG | susceptibility | 1.35 | 0.12 |
| | | | AA | (protective) | | |
| Galectin-2 (LGALS2) | C3279T | 7291467 | GG | protective | 0.65 | 0.12 |

### Discussion

The above results show that several polymorphisms were associated with either increased or decreased risk of developing ACS. The associations of individual polymorphisms on their own, while of discriminatory value, are sometimes unlikely to offer an acceptable prediction of disease. However, in combination these polymorphisms distinguish susceptible subjects from those who are resistant (for example, between the smokers who develop ACS and those with the least risk with comparable smoking exposure). The polymorphisms represent both promoter polymorphisms, thought to modify gene expression and hence protein synthesis, and exonic polymorphisms known to alter amino-acid sequence (and likely expression and/or function) in a number of genes encoding proteins central to processes including inflammation, matrix remodelling, and cytokine activity.

In the comparison of smokers with ACS and matched smokers without ACS (lowest risk for ACS despite smoking), several polymorphisms were identified as being found in significantly greater or lesser frequency than in the comparator group. Due to the small cohort of ACS patients, polymorphisms where there are only trends towards differences (P=0.06-0.25) were included in the analyses, although in the combined analyses only those polymorphisms with the most significant differences were utilized.
In the analysis of the Y402H C/T polymorphism in the gene encoding Complement factor H, the TT genotype was found to be greater in the ACS cohort compared to resistant smoker cohort (OR=1.5, p=0.04) consistent with a susceptibility role (see Table 3).
In the analysis of the Asp92Asn A/G polymorphism in the gene encoding Myeloid IgA Fc receptor, the AA and AG genotypes were found to be greater in the resistant smoker cohort compared to the ACS cohort (OR=0.63, p=0.13) consistent with each having a protective role (see Table 4). In contrast the GG genotype was found to be consistent with a susceptibility role (see Table 4).
In the analysis of the A387P C/G polymorphism in the gene encoding Thrombospondin 4, the GG genotype was found to be greater in the resistant smoker cohort compared to the ACS cohort (OR=0.36, p=0.05) consistent with a protective role (see Table 5). The G allele was also found to be significantly greater in the resistant smoker cohort compared to the ACS cohort (OR=0.72, p=0.04) consistent with a protective role (see Table 5).
In the analysis of the A/G (rs4804611) polymorphism in the gene encoding Zinc finger protein 627, the GA and GG genotypes were each found to be greater ACS cohort compared to the resistant smoker cohort (OR=1.63, p=0.01) consistent with each having a susceptibility role (see Table 6). The G allele was also found to be greater in the ACS cohort compared to the resistant smoker cohort (OR=1.49, p=0.07) consistent with a susceptibility role. In contrast the AA genotype was found to be consistent with a protective role (see Table 6).
In the Asp51Ala A/C polymorphism in the gene encoding Interleukin 1 family member 10, the CC genotype was found to be greater in the ACS cohort compared to the resistant smoker cohort (OR=2.10, p=0.003) consistent with a susceptibility role (see Table 7). The C allele was also found to be greater in the ACS cohort compared to the resistant smoker cohort (OR=1.25, p=0.10) consistent with a susceptibility role (see Table 7).
In the Asn159Asn A/G polymorphism in the gene encoding Serpin 2, the AG and GG genotypes were each found to be greater than the ACS cohort compared to the resistant smoker cohort (OR=1.35, p=0.12) consistent with each having a susceptibility role (see Table 8). In contrast the AA genotype was found to be consistent with a protective role (see Table 8).
In the analysis of the C3279T A/G polymorphism in the gene encoding Galectin-2, the GG genotype was found to be greater in the resistant smoker cohort compared to the ACS cohort (OR=0.65, p=0.12) consistent with a protective role (see Table 9). The G allele was also found to be greater in the resistant smoker cohort compared to the ACS cohort (OR=0.78, p=0.07) consistent with a protective role (see Table 9).

It is accepted that the disposition to ACS is the result of the combined effects of the individual's genetic makeup and other factors, including their lifetime exposure to various aero-pollutants including tobacco smoke. Similarly, it is accepted that ACS encompasses several vascular diseases. The data herein suggest that several genes can contribute to the development of ACS. A number of genetic mutations working in combination either promoting or protecting the vasculature from damage are likely to be involved in elevated resistance or susceptibility to ACS.

From the analyses of the individual polymorphisms, 5 susceptibility genotypes and 5 protective genotypes were identified and analyzed for their frequencies in the smoker cohort consisting of resistant smokers and those with ACS. In a pre-defined algorithm, where the presence of a susceptibility genotype scores +1 and the presence of a protective genotype scores −1, an ACS SNP score can be generated for each subject. The ACS SNP score generated with reference to a SNP panel can then be related to the frequency of having ACS.

The ACS SNP score can be independently associated with having ACS and can be used alone or in conjunction with non-genetic risk factors to assess risk of ACS, arterial inflammation, or ACS-associated impaired vascular function and of having an acute coronary event.

These findings indicate that the methods of the present invention may be predictive of ACS in an individual well before symptoms present.

These findings therefore also present opportunities for therapeutic interventions and/or treatment regimens, as discussed herein. Briefly, such interventions or regimens can include the provision to the subject of motivation to implement a lifestyle change, or therapeutic methods directed at normalizing aberrant gene expression or gene product function. For example, the genotypes AA and AB are associated with decreased risk of developing ACS, while the BB genotype is associated with increased risk of developing ACS. The A allele is reportedly associated with increased binding of a repressor protein and decreased transcription of the gene. A suitable therapy for individuals having the BB genotype can be the administration of an agent capable of increasing the level of repressor and/or enhancing binding of the repressor, thereby augmenting its downregulatory effect on transcription. An alternative therapy can include gene therapy, for example the introduction of at least one additional copy of a gene encoding a repressor having an increased affinity for binding a gene having a BB genotype.

In another example, a given susceptibility genotype is associated with increased expression of a gene relative to that observed with the protective genotype. A suitable therapy in subjects known to possess the susceptibility genotype is the administration of an agent capable of reducing expression of the gene, for example using antisense or RNAi methods. An alternative suitable therapy can be the administration to such a subject of an inhibitor of the gene product. In still another example, a susceptibility genotype present in the promoter of a gene is associated with increased binding of a repressor protein and decreased transcription of the gene. A suitable therapy is the administration of an agent capable of decreasing the level of repressor and/or preventing binding of the repressor, thereby alleviating its downregulatory effect on transcription. An alternative therapy can include gene therapy, for example the introduction of at least one additional copy of the gene having a reduced affinity for repressor binding (for example, a gene copy having a protective genotype).

Suitable methods and agents for use in such therapy are well known in the art, and are discussed herein.

The identification of both susceptibility and protective polymorphisms as described herein also provides the opportunity to screen candidate compounds to assess their efficacy in methods of prophylactic and/or therapeutic treatment. Such screening methods involve identifying which of a range of candidate compounds have the ability to reverse or counteract a genotypic or phenotypic effect of a susceptibility polymorphism, or the ability to mimic or replicate a genotypic or phenotypic effect of a protective polymorphism.

Still further, methods for assessing the likely responsiveness of a subject to an available prophylactic or therapeutic approach are provided. Such methods have particular application where the available treatment approach involves restoring the physiologically active concentration of a product of an expressed gene from either an excess or deficit to be within a range which is normal for the age and sex of the subject. In such cases, the method comprises the detection of the presence or absence of a susceptibility polymorphism which when present either upregulates or downregulates expression of the gene such that a state of such excess or deficit is the outcome, with those subjects in which the polymorphism is present being likely responders to treatment.

Table 11 below presents representative examples of polymorphisms in linkage disequilibrium with the polymorphisms specified herein in Table 10. Examples of such polymorphisms can be located using public databases, such as that available at www.hapmap.org. Specified polymorphisms are indicated in bold. As those skilled in the art will recognize, the rs numbers provided are identifiers unique to each polymorphism.

These results show that SNPs in LD with the SNPs recited herein, such as those from Table 11, could be utilized in a SNP score with similar clinical utility.

TABLE 11
SNPs in linkage disequilibrium with the SNPs associated with either a
susceptibility or protective phenotype.
CFH

| | | | | | | | |
|---|---|---|---|---|---|---|---|
| rs9658961 | rs12124794 | rs5779847 | rs7514261 | rs460897 | rs1082871 | rs420553 | rs529825 |
| rs12405238 | rs35571081 | rs380390 | rs460184 | rs1082872 | rs35850052 | rs35196104 | rs12136675 |
| rs34395480 | rs380060 | rs28929497 | rs1082873 | rs10922115 | rs34902514 | rs12040718 | rs3043112 |
| rs7540032 | rs463726 | rs420523 | rs11807997 | rs34388368 | rs10922095 | rs3043113 | rs10922108 |
| rs14473 | rs408143 | rs36040881 | rs6660100 | rs10922096 | rs28613548 | rs414539 | rs459598 |
| rs1092801 | rs35104148 | rs1156679 | rs12030500 | rs3043115 | rs2284664 | rs35742764 | rs549999 |
| rs369561 | rs1156678 | rs3645 | rs7415913 | rs1329428 | rs488738 | rs1082874 | rs385390 |
| rs10616982 | rs12041668 | rs7413999 | rs2284663 | rs12756364 | rs506584 | rs4997205 | rs35050365 |
| rs518572 | rs2878647 | rs7413137 | rs386258 | rs507384 | rs446868 | rs11809183 | rs35885828 |
| rs5779848 | rs395963 | rs1089031 | rs1082875 | rs4997206 | rs36014405 | rs12032372 | rs34876440 |
| rs412852 | rs800269 | rs426566 | rs4997207 | rs567284 | rs514943 | rs5022897 | rs35253683 |
| rs550116 | rs1754452 | rs4997208 | rs485155 | rs7546015 | rs5022898 | rs10801559 | rs550147 |
| rs426330 | rs454834 | rs36049876 | rs1089038 | rs5022899 | rs2064456 | rs550861 | rs510059 |
| rs383372 | rs6691749 | rs12033127 | rs5022900 | rs1329427 | rs506342 | rs800238 | rs35703353 |
| rs514591 | rs10922097 | rs5022901 | rs10922109 | rs506317 | rs522401 | rs35267550 | rs35107961 |
| rs488380 | rs4350148 | rs35878624 | rs2936006 | rs568588 | rs35459176 | rs11579439 | rs579745 |
| rs6685249 | rs70620 | rs385259 | rs448696 | rs34265062 | rs35566996 | rs10922098 | rs10685027 |
| rs70621 | rs34110598 | rs776062 | rs35609786 | rs35291271 | rs485632 | rs203676 | rs731557 |
| rs384940 | rs776063 | rs34286646 | rs10664537 | rs10922099 | rs35876902 | rs434536 | rs384837 |
| rs444295 | rs34408013 | rs36042724 | rs10922100 | rs4044882 | rs742855 | rs459597 | rs411729 |
| rs35774441 | rs5779844 | rs12038674 | rs203675 | rs374231 | rs33952268 | rs445568 | rs364320 |
| rs34111659 | rs1292473 | rs35688523 | rs34789365 | rs33982034 | rs412632 | rs12748435 | rs4044888 |
| rs1292472 | rs6677089 | rs435628 | rs456474 | rs776067 | rs12723496 | rs16840401 | rs28853072 |
| rs35216365 | rs375046 | rs461875 | rs776068 | rs12748610 | rs34327103 | rs7539005 | rs6688272 |
| rs35945332 | rs403990 | rs776069 | rs35714451 | rs35636447 | rs529899 | rs6664877 | rs428060 |
| rs2133143 | rs776070 | rs35001925 | rs35661539 | rs11580821 | rs6677460 | rs3753397 | rs35866667 |
| rs575986 | rs434491 | rs34731535 | rs10922102 | rs35031568 | rs34748127 | rs776100 | rs474300 |
| rs424878 | rs551397 | rs2860102 | rs203674 | rs35292876 | rs776099 | rs488481 | rs376498 |
| rs800292 | rs34813609 | rs35453854 | rs515299 | rs33935994 | rs776072 | rs376515 | rs34895813 |
| rs10801557 | rs35806886 | rs11799956 | rs33977802 | rs11805258 | rs376841 | rs559350 | rs5003626 |
| rs12085209 | rs34344258 | rs402032 | rs490415 | rs425524 | rs35284444 | rs5003625 | rs16840462 |
| rs543879 | rs474132 | rs449847 | rs11806293 | rs35507625 | rs5003624 | rs34938865 | rs35700477 |
| rs776098 | rs425173 | rs35151217 | rs35814900 | rs4658046 | rs35361417 | rs34807691 | rs776097 |
| rs491400 | rs28363723 | rs495222 | rs10754199 | rs34622202 | rs422682 | rs776096 | rs1754450 |
| rs5779849 | rs34351402 | rs10922103 | rs10754200 | rs35331736 | rs776095 | rs1754449 | rs378940 |
| rs34181066 | rs28664709 | rs16840465 | rs412739 | rs388116 | rs444476 | rs435153 | rs34842495 |
| rs35475334 | rs34274678 | rs408497 | rs466638 | rs776078 | rs5007012 | rs35108970 | rs34230295 |
| rs35661772 | rs34745219 | rs776092 | rs800232 | rs5007013 | rs16840410 | rs10536523 | rs35759609 |
| rs34752546 | rs460376 | rs10801562 | rs5007014 | rs1329424 | rs35549235 | rs3753395 | rs35279122 |
| rs1082900 | rs1092228 | rs5007015 | rs572515 | rs10540668 | rs6677604 | rs11799380 | rs502202 |
| rs1089025 | rs5007016 | rs1329423 | rs12565418 | rs34900334 | rs454652 | rs36040396 | rs1089024 |
| rs5007017 | rs34050381 | rs368465 | rs10465586 | rs34279302 | rs1082898 | rs28470810 | rs5779850 |
| rs3766403 | rs402056 | rs10489456 | rs407361 | rs440950 | rs401473 | rs5007018 | rs34940854 |
| rs203688 | rs10922104 | rs34794150 | rs440828 | rs10922110 | rs5007019 | rs34683486 | rs12038333 |
| rs203673 | rs405306 | rs1082895 | rs10922111 | rs388419 | rs34228611 | rs12045503 | rs2104714 |
| rs2173383 | rs1082894 | rs10922112 | rs449657 | rs34239310 | rs2268343 | rs10465603 | rs34932940 |
| rs1082893 | rs10922113 | rs17575274 | rs12116702 | rs9970075 | rs203672 | rs2336221 | rs1082892 |
| rs383961 | rs620015 | rs12127759 | rs9970784 | rs203671 | rs34137380 | rs1082891 | rs470182 |
| rs34214907 | rs34028773 | rs1831282 | rs203670 | rs35742991 | rs504884 | rs374823 | rs445207 |
| rs35780892 | rs203687 | rs1587325 | rs424535 | rs1082890 | rs421480 | rs409582 | rs35855516 |
| rs2019727 | rs203669 | rs34557289 | rs527488 | rs421440 | rs568860 | rs17574369 | rs2019724 |
| rs6682138 | rs1065489 | rs1082889 | rs373453 | rs568178 | rs766001 | rs1048663 | rs10922105 |
| rs11582939 | rs1082888 | rs391423 | rs568121 | rs3834020 | rs1887973 | rs33956114 | rs35935657 |
| rs1082887 | rs1089023 | rs12397458 | rs3043111 | rs2300429 | rs203668 | rs385892 | rs1082886 |
| rs382345 | rs566159 | rs34086255 | rs6428357 | rs10922106 | rs16840522 | rs529541 | rs1089022 |
| rs34452879 | rs35121684 | rs7513157 | rs12402808 | rs385543 | rs1082885 | rs381383 | rs401216 |
| rs34543613 | rs35788722 | rs12025861 | rs17575212 | rs1082884 | rs433349 | rs401161 | rs34473169 |
| rs6695321 | rs11801630 | rs534399 | rs12759472 | rs380733 | rs370789 | rs16840419 | rs402991 |
| rs374896 | rs11539862 | rs1082883 | rs435290 | rs421820 | rs3766404 | rs399469 | rs12047565 |
| rs34362004 | rs35174779 | rs2772036 | rs391537 | rs34727645 | rs34916950 | rs393955 | rs1040597 |
| rs1082882 | rs4322183 | rs401188 | rs35756883 | rs34356041 | rs34831442 | rs34594237 | rs380296 |
| rs4287123 | rs390154 | rs33944729 | rs203686 | rs35566405 | rs35496304 | rs1091359 | rs4539076 |
| rs400642 | rs35449482 | rs33915960 | rs381974 | rs395129 | rs379980 | rs422273 | rs400344 |
| rs33982697 | rs10733086 | rs34699290 | rs466287 | rs1082880 | rs405269 | rs5002709 | rs16840422 |
| rs1410997 | rs35908703 | rs460787 | rs566881 | rs800228 | rs5002710 | rs35198449 | rs5014740 |
| rs35717509 | rs2746965 | rs2772038 | rs404088 | rs5002711 | rs35462027 | rs5014739 | rs35582046 |
| rs1984894 | rs458022 | rs429123 | rs5002712 | rs1061147 | rs5014738 | rs35612319 | rs35194983 |
| rs466540 | rs800227 | rs5002713 | rs35097611 | rs5014737 | rs35828462 | rs36072242 | rs456190 |
| rs1831273 | rs5002714 | rs35225053 | rs5014736 | rs12096637 | rs513699 | rs11585571 | rs395591 |
| rs5002715 | rs34137105 | rs5014735 | rs36014159 | rs35274867 | rs1066423 | rs422992 | rs387111 |
| rs490864 | rs5014734 | rs379489 | rs35343172 | rs466501 | rs422795 | rs387107 | rs34639660 |
| rs5014733 | rs34853939 | rs17434860 | rs1066422 | rs1831272 | rs386185 | rs35285703 | rs11398897 |
| rs1474792 | rs409953 | rs1066421 | rs1754446 | rs439365 | rs1329422 | rs6664705 | rs34697646 |
| rs464798 | rs11580690 | rs1754445 | rs378283 | rs35108279 | rs12406047 | rs35505017 | rs35352142 |
| rs1066420 | rs1754444 | rs389897 | rs34058609 | rs203685 | rs35206437 | rs422851 | rs776089 |
| rs421581 | rs377298 | rs514756 | rs203684 | rs34436878 | rs430173 | rs11580699 | rs434419 |
| rs384032 | rs3216571 | rs203683 | rs28442192 | rs35935173 | rs11585965 | rs10661231 | rs2473994 |
| rs34386071 | rs7522681 | rs3766405 | rs2020130 | rs776088 | rs2336471 | rs374905 | rs2300430 |
| rs383191 | rs35479160 | rs34347090 | rs1280511 | rs454085 | rs427939 | rs10801553 | rs398248 |
| rs34763899 | rs35462210 | rs1280510 | rs454005 | rs3073685 | rs1329421 | rs2772040 | rs3753396 |
| rs35052326 | rs776087 | rs413384 | rs10922120 | rs34860966 | rs12047103 | rs35870521 | rs2336222 |
| rs1292421 | rs1854499 | rs12738227 | rs544889 | rs12039905 | rs34193797 | rs2878648 | rs466800 |
| rs453912 | rs12723806 | rs34853086 | rs12047106 | rs765774 | rs2878649 | rs776085 | rs3925263 |
| rs643781 | rs34328658 | rs203682 | rs7537967 | rs2336223 | rs462795 | rs34419350 | rs12738240 |
| rs34219315 | rs10737679 | rs7535653 | rs2336224 | rs776083 | rs12566207 | rs367684 | rs11318544 |
| rs2772039 | rs35762927 | rs422404 | rs776082 | rs476521 | rs12723972 | rs570618 | rs203681 |
| rs34974223 | rs10801560 | rs776081 | rs452284 | rs12738599 | rs35063447 | rs10737680 | rs403846 |
| rs10801561 | rs460232 | rs34749367 | rs426736 | rs10922092 | rs35617250 | rs35626603 | rs35866386 |
| rs455497 | rs119024 | rs10801554 | rs34845806 | rs35634602 | rs36082199 | rs776079 | rs119023 |
| rs12069060 | rs11584505 | rs419137 | rs34247141 | rs9427627 | rs12568400 | rs34125349 | rs5002874 |
| rs1410996 | rs491480 | rs12138995 | rs12039050 | rs7529589 | rs5779845 | rs35263559 | rs34231058 |
| rs460534 | rs12046285 | rs482934 | rs5002875 | rs34799930 | rs33968127 | rs460481 | rs34130738 |
| rs28397680 | rs5002876 | rs36024842 | rs11339120 | rs430164 | rs369816 | rs35695425 | rs5002877 |
| rs1329429 | rs36054875 | rs460306 | rs35191813 | rs12029785 | rs5779846 | rs1060821 | rs519839 |
| rs498492 | rs510755 | rs34815383 | rs5002878 | rs35537678 | rs518957 | rs800243 | rs366162 |
| rs1061170 | rs5002879 | rs34018998 | rs105980 | rs466344 | rs2878713 | rs34331968 | rs5002880 |
| rs34420836 | rs495968 | rs456761 | rs366818 | rs36062459 | rs1831281 | rs395544 | rs34802957 |
| rs443134 | rs11807686 | rs34705877 | rs12134598 | rs436337 | rs34813995 | rs456243 | rs367258 |
| rs10801555 | rs203680 | rs34999101 | rs4044884 | rs466553 | rs395998 | rs10801556 | rs12042805 |
| rs6689009 | rs35075161 | rs34666176 | rs373317 | rs4657826 | rs7535263 | rs10922107 | rs420922 |
| rs800241 | rs385532 | rs12726401 | rs203679 | rs34734075 | rs420921 | rs466405 | rs445413 |
| rs12740961 | rs2274700 | rs364947 | rs35732058 | rs434099 | rs11584932 | rs34488706 | rs34399588 |
| rs1576340 | rs409319 | rs776057 | rs34422022 | rs34202669 | rs1061171 | rs12144939 | rs409308 |
| rs453645 | rs12408446 | rs528298 | rs35923803 | rs10801558 | rs493367 | rs1066415 | rs538113 |
| rs10922093 | rs203678 | rs11799595 | rs536564 | rs1082869 | rs10922114 | rs35397685 | rs1831280 |
| rs371647 | rs536539 | rs401808 | rs7412846 | rs10922094 | rs203677 | rs35952524 | rs9427909 |
| rs427997 | rs7412847 | | | | | | |

FCAR

| | | | | | | | |
|---|---|---|---|---|---|---|---|
| rs3826866 | rs35886422 | rs12151256 | rs12980503 | rs640345 | rs13345741 | rs2365579 | rs3826867 |
| rs2966884 | rs11672006 | rs28754932 | rs3745892 | rs12459411 | rs12976082 | rs3826868 | rs35496566 |
| rs11672012 | rs11883076 | rs667271 | rs17771967 | rs12976517 | rs3826869 | rs10402857 | rs11672015 |
| rs11883020 | rs668655 | rs35676399 | rs12976533 | rs2966886 | rs34242342 | rs11667722 | rs11883080 |
| rs3745893 | rs35959167 | rs16986050 | rs2966885 | rs625698 | rs11667798 | rs11883047 | rs34068780 |
| rs10421406 | rs10413148 | rs678812 | rs625718 | rs11667799 | rs34647213 | rs654686 | rs10421822 |
| rs10414707 | rs678846 | rs35081623 | rs34177062 | rs6509902 | rs2043329 | rs35935247 | rs4806611 |
| rs35989363 | rs12973384 | rs4806452 | rs11666074 | rs655534 | rs11672983 | rs7249884 | rs35747711 |
| rs470945 | rs4806453 | rs12608589 | rs1743322 | rs17771979 | rs34909097 | rs581623 | rs34370232 |
| rs2916049 | rs11878537 | rs34354985 | rs11665986 | rs10603427 | rs682148 | rs470835 | rs3097897 |
| rs4806592 | rs2966840 | rs17781556 | rs7253001 | rs35545130 | rs34997427 | rs2916050 | rs4806593 |
| rs685084 | rs11666055 | rs12981397 | rs4247375 | rs2295804 | rs2916051 | rs4806594 | rs3745894 |
| rs11666065 | rs35107550 | rs11396353 | rs2295805 | rs28453291 | rs28484282 | rs1048270 | rs6509904 |
| rs35326923 | rs35802190 | rs2916038 | rs28590562 | rs8105869 | rs1048271 | rs17836457 | rs2365580 |
| rs585742 | rs2966882 | rs12151085 | rs28513532 | rs3745896 | rs11084374 | rs4531854 | rs35443733 |
| rs2966881 | rs10423866 | rs663815 | rs592446 | rs17772004 | rs3032893 | rs597013 | rs638584 |
| rs4563149 | rs10567528 | rs605746 | rs12462181 | rs4310985 | rs34253442 | rs2916039 | rs4575639 |
| rs655687 | rs35275981 | rs3816051 | rs4305197 | rs34583400 | rs2916041 | rs5011102 | rs12460473 |
| rs10604255 | rs2304225 | rs7507282 | rs34986537 | rs1654641 | rs5828606 | rs4806595 | rs4806597 |
| rs11084375 | rs7507269 | rs605219 | rs3826865 | rs5011103 | rs8109630 | rs35844018 | rs11084376 |
| rs4806612 | rs12975219 | rs1654642 | rs5011104 | rs2984177 | rs621712 | rs11084377 | rs35092488 |
| rs598375 | rs640396 | rs5011105 | rs10719073 | rs624783 | rs8112766 | rs4806613 | rs4806449 |
| rs3826870 | rs5828607 | rs2984179 | rs36085502 | rs12461607 | rs35177585 | rs606225 | rs3842418 |
| rs5011106 | rs2984180 | rs35625604 | rs10451424 | rs35509168 | rs4806450 | rs640445 | rs6146558 |
| rs34180457 | rs11668926 | rs10407012 | rs34882261 | rs10664307 | rs34892101 | rs11881042 | rs34197131 |
| rs663812 | rs4806601 | rs35124662 | rs35240925 | rs3826872 | rs3885185 | rs9749587 | rs35521613 |
| rs36005625 | rs35157065 | rs10666144 | rs34697590 | rs4560031 | rs34330719 | rs651995 | rs4806602 |
| rs12460405 | rs34003399 | rs3826873 | rs3885184 | rs12461104 | rs35733063 | rs4806603 | rs10416381 |
| rs35658498 | rs640854 | rs4541181 | rs35628894 | rs678675 | rs28642682 | rs10416385 | rs35286779 |
| rs35667877 | rs4474811 | rs9749595 | rs34254306 | rs28536683 | rs10416213 | rs34831605 | rs3826874 |
| rs4541182 | rs9749600 | rs34757959 | rs35960065 | rs34607125 | rs611763 | rs35915433 | rs4446002 |
| rs9749607 | rs654255 | rs4806604 | rs10416940 | rs34687898 | rs3826877 | rs4806583 | rs35152131 |
| rs35723337 | rs28897069 | rs12462511 | rs12983499 | rs35897626 | rs4806584 | rs3865512 | rs35343287 |
| rs7253636 | rs12462528 | rs34170735 | rs653019 | rs4806585 | rs28373134 | rs34882931 | rs35302726 |
| rs12460479 | rs613491 | rs34891547 | rs34918222 | rs35572033 | rs3930237 | rs4806605 | rs12462499 |
| rs620977 | rs34969817 | rs11084370 | rs671600 | rs35560234 | rs7257926 | rs12462519 | rs4080176 |
| rs662994 | rs11882549 | rs35902110 | rs586955 | rs7246086 | rs4299267 | rs614891 | rs35188903 |
| rs11084371 | rs2365252 | rs680297 | rs10402725 | rs12459447 | rs34626017 | rs12974193 | rs11882616 |
| rs34826002 | rs34597621 | rs10402743 | rs4474809 | rs615341 | rs12973588 | rs12983174 | rs35440472 |
| rs4806598 | rs8100793 | rs6509908 | rs35492675 | rs2916045 | rs11673300 | rs35960226 | rs600888 |
| rs34625687 | rs7253995 | rs3189235 | rs2916046 | rs11673276 | rs621019 | rs10407958 | rs8101852 |
| rs34840655 | rs642893 | rs12974749 | rs35944751 | rs2365253 | rs601838 | rs12608573 | rs10421219 |
| rs642941 | rs12976350 | rs4346307 | rs621924 | rs4806599 | rs8101381 | rs35582928 | rs643347 |
| rs36033968 | rs4413089 | rs2261769 | rs4806454 | rs8101702 | rs8107890 | rs1049150 | rs7255036 |
| rs11084372 | rs1654643 | rs11347116 | rs28880098 | rs10421281 | rs643861 | rs28498203 | rs2966873 |
| rs1743319 | rs11347115 | rs6509905 | rs8111377 | rs4806568 | rs28374872 | rs2916052 | rs607380 |
| rs10407172 | rs10406079 | rs11327547 | rs4806569 | rs12981060 | rs2916053 | rs2886079 | rs17814543 |
| rs10423668 | rs4806459 | rs2273730 | rs28382394 | rs2916054 | rs607382 | rs34556293 | rs35282099 |
| rs4806614 | rs1065331 | rs12982007 | rs12459407 | rs1743320 | rs35429338 | rs7259090 | rs35171123 |
| rs660405 | rs28522319 | rs1130479 | rs1743321 | rs35631470 | rs7259347 | rs34614852 | rs2273731 |
| rs3097896 | rs1143507 | rs608287 | rs12610372 | rs7247521 | rs35358533 | rs36097059 | rs4806572 |
| rs1049284 | rs1130471 | rs34848245 | rs7248382 | rs4806460 | rs34827252 | rs35970023 | rs3189394 |
| rs2364464 | rs35946352 | rs7247547 | rs34653350 | rs4806573 | rs1130480 | rs34450084 | rs12608797 |
| rs12975418 | rs671925 | rs4239590 | rs1130481 | rs622363 | rs12608799 | rs34764559 | rs665101 |
| rs4806574 | rs1130482 | rs10421802 | rs12608800 | rs9797555 | rs673316 | rs4806575 | rs3206658 |
| rs623167 | rs34840288 | rs4239591 | rs35020315 | rs34989611 | rs3189398 | rs3810347 | rs35005744 |
| rs34472333 | rs674268 | rs35080576 | rs1130485 | rs34247664 | rs616452 | rs8102504 | rs674712 |
| rs4806576 | rs1130486 | rs3810348 | rs616577 | rs4806606 | rs12461010 | rs34727739 | rs1130487 |
| rs2916056 | rs1654644 | rs11671260 | rs34649375 | rs35610427 | rs1143508 | rs28670652 | rs1987051 |
| rs10412499 | rs35746443 | rs35608990 | rs1130489 | rs650391 | rs12981377 | rs11671686 | rs687844 |
| rs35360058 | rs1130491 | rs2916057 | rs12979452 | rs10418998 | rs688250 | rs35604903 | rs2966872 |
| rs2966888 | rs12980151 | rs12977049 | rs688276 | rs4806577 | rs10406301 | rs2966887 | rs7507739 |
| rs12978928 | rs1049209 | rs4806578 | rs1130492 | rs3810345 | rs35461725 | rs12978955 | rs1049215 |
| rs4806579 | rs35360844 | rs34397737 | rs4806600 | rs28756208 | rs34481025 | rs1130466 | rs4806586 |
| rs651820 | rs35043300 | rs4806607 | rs35974949 | rs1130467 | rs10413739 | rs10422740 | rs4487030 |
| rs4806608 | rs34640119 | rs1130468 | rs34722682 | rs2004717 | rs4806456 | rs7260414 | rs583070 |
| rs9676587 | rs17739894 | rs34411298 | rs4806457 | rs4806609 | rs35124837 | rs2916047 | rs12460121 |
| rs35310125 | rs4806458 | rs34391636 | rs594307 | rs5020578 | rs4806587 | rs35070447 | rs3826878 |
| rs10401687 | rs34348626 | rs2966878 | rs4806588 | rs4020166 | rs2915985 | rs35717373 | rs35296616 |
| rs34775109 | rs11880061 | rs10522239 | rs2915986 | rs10402324 | rs35668498 | rs2916048 | rs4806589 |
| rs35189301 | rs2915987 | rs1865096 | rs35666737 | rs1130472 | rs11269227 | rs35908355 | rs2915988 |
| rs11666735 | rs596692 | rs1130473 | rs35757649 | rs610710 | rs3745897 | rs1865097 | rs597500 |
| rs1130513 | rs4806590 | rs680377 | rs10417848 | rs11666846 | rs34092079 | rs1130515 | rs4806591 |
| rs636821 | rs3745898 | rs12974020 | rs34409000 | rs1130516 | rs3189418 | rs3952577 | rs3745899 |
| rs35182606 | rs8109574 | rs1049259 | rs11880084 | rs2365223 | rs3745900 | rs12974530 | rs35612937 |
| rs1049271 | rs1130503 | rs34020429 | rs3745901 | rs12972637 | rs3898893 | rs12985492 | rs11880090 |
| rs611728 | rs3745902 | rs12975083 | rs3898894 | rs2955 | rs1049290 | rs34247194 | rs3745903 |
| rs28542649 | rs35010614 | rs2954 | rs1130504 | rs612143 | rs2966890 | rs7258735 | rs615169 |
| rs4806580 | rs1130505 | rs2915976 | rs2915989 | rs28529432 | rs4806571 | rs4806581 | rs34984350 |
| rs2915977 | rs2915990 | rs28642207 | rs2916036 | rs4806582 | rs34942754 | rs652188 | rs2915991 |
| rs7258306 | rs4806451 | rs5828604 | rs34465199 | rs35336813 | rs2915992 | rs7258679 | rs2916037 |
| rs5828605 | rs12980633 | rs7259988 | rs28533724 | rs12460904 | rs1654640 | rs12150998 | rs12462968 |
| rs639850 | rs1865095 | rs4560030 | | | | | |

THBS4

| | | | | | | |
|---|---|---|---|---|---|---|
| rs35831290 | rs2438603 | rs445471 | rs34891970 | rs6889033 | rs34961504 | rs17878919 |
| rs17880390 | rs9293800 | rs34347757 | rs2545122 | rs17878697 | rs17879615 | rs17882372 |
| rs34385440 | rs2434307 | rs414797 | rs17879362 | rs17885704 | rs10553459 | rs6878861 |
| rs3813667 | rs17879218 | rs11343128 | rs3991743 | rs2434308 | rs4425490 | rs404375 |
| rs17885865 | rs5869018 | rs34258045 | rs17878424 | rs17885225 | rs2241824 | rs2247450 |
| rs2438618 | rs3749684 | rs17882273 | rs12659471 | rs10643041 | rs2434309 | rs17886956 |
| rs13174295 | rs4703797 | rs34583152 | rs2434310 | rs17882731 | rs35683982 | rs1465853 |
| rs7714280 | rs2451932 | rs17885143 | rs5869016 | rs17883112 | rs10657162 | rs2434316 |
| rs17882223 | rs1438737 | rs4345304 | rs2118732 | rs34366253 | rs17884706 | rs6897811 |
| rs17880018 | rs12656480 | rs2438617 | rs17879904 | rs35422105 | rs17879800 | rs13158203 |
| rs2438616 | rs17879695 | rs17879094 | rs11377619 | rs12656513 | rs11408457 | rs17886538 |
| rs1438736 | rs34117433 | rs10673146 | rs7721411 | rs2059794 | rs1438735 | rs256439 |
| rs36098825 | rs2438615 | rs11739940 | rs35597508 | rs256438 | rs12109615 | rs34387198 |
| rs2438651 | rs6861685 | rs17882708 | rs34307157 | rs12332358 | rs17885055 | rs364988 |
| rs16877469 | rs12109181 | rs6870882 | rs11738491 | rs10474605 | rs35849766 | rs7707343 |
| rs2451933 | rs17886031 | rs382746 | rs28628197 | rs11393694 | rs6878264 | rs17881847 |
| rs17882167 | rs35973285 | rs10713901 | rs2438614 | rs11741724 | rs435610 | rs6874418 |
| rs35357036 | rs35650587 | rs17878628 | rs17883722 | rs34506854 | rs2438644 | rs2028269 |
| rs17878376 | rs368287 | rs35289764 | rs2438643 | rs2438613 | rs17886994 | rs426623 |
| rs6889646 | rs6453500 | rs2434317 | rs36080988 | rs17879373 | rs17885132 | rs2434305 |
| rs6870639 | rs13181102 | rs412379 | rs17882230 | rs6453501 | rs2438612 | rs17882422 |
| rs423906 | rs256437 | rs7727310 | rs2434318 | rs2434311 | rs17882513 | rs3217460 |
| rs2918423 | rs16877428 | rs17885466 | rs438042 | rs17882916 | rs35953385 | rs34882587 |
| rs17881955 | rs405482 | rs12110039 | rs11462765 | rs34870929 | rs17879921 | rs447875 |
| rs3749685 | rs34886525 | rs2434319 | rs17879633 | rs17878812 | rs7711310 | rs7716835 |
| rs2451940 | rs17879415 | rs17878515 | rs12659722 | rs2434279 | rs2438611 | rs411240 |
| rs407314 | rs17878747 | rs13156952 | rs2434320 | rs440272 | rs6874882 | rs13167730 |
| rs6859206 | rs256449 | rs398774 | rs17885895 | rs35229148 | rs2438642 | rs2172093 |
| rs366553 | rs405112 | rs394947 | rs2434280 | rs35373315 | rs2438650 | rs17880078 |
| rs35937190 | rs2438641 | rs5869015 | rs35901096 | rs10474606 | rs6897999 | rs35810553 |
| rs35859021 | rs256448 | rs397601 | rs10035503 | rs2438640 | rs256450 | rs256447 |
| rs2405136 | rs17885484 | rs2434281 | rs256451 | rs17882488 | rs2249687 | rs12514383 |
| rs2438639 | rs34704233 | rs17879105 | rs692979 | rs384941 | rs2438638 | rs2434270 |
| rs35811803 | rs2249794 | rs17882585 | rs7710472 | rs2434271 | rs194375 | rs693270 |
| rs2288394 | rs35351529 | rs11743110 | rs34535741 | rs690284 | rs17883913 | rs2438637 |
| rs747099 | rs256446 | rs12519402 | rs1130758 | rs2434282 | rs11954663 | rs11362890 |
| rs34349294 | rs2229398 | rs2434283 | rs13154936 | rs166270 | rs35304250 | rs1049798 |
| rs2438636 | rs2118731 | rs256445 | rs368936 | rs17880024 | rs34836557 | rs6875852 |
| rs256444 | rs2241826 | rs17879739 | rs34338186 | rs12234104 | rs256443 | rs2241825 |
| rs17879514 | rs11462770 | rs2434272 | rs256442 | rs3214681 | rs10037941 | rs35852100 |
| rs13188176 | rs17885154 | rs35303028 | rs5869017 | rs34655435 | rs2438610 | rs256441 |
| rs432267 | rs3214550 | rs2434284 | rs34851741 | rs12332694 | rs411943 | rs17885983 |
| rs2434285 | rs385771 | rs17884143 | rs434409 | rs2288395 | rs2438635 | rs366471 |
| rs17879871 | rs2434304 | rs17878910 | rs2438634 | rs2434273 | rs256440 | rs35707304 |
| rs10514175 | rs12523107 | rs2434274 | rs17886500 | rs2434303 | rs34314822 | rs12523112 |
| rs2434275 | rs17886383 | rs34023954 | rs17883166 | rs2438633 | rs6874832 | rs34179843 |
| rs2434302 | rs2434301 | rs2434286 | rs2913545 | rs13171081 | rs401302 | rs35977043 |
| rs2434287 | rs2438609 | rs690325 | rs34249634 | rs6891246 | rs2438632 | rs34015132 |
| rs7723567 | rs17882621 | rs17878992 | rs13153268 | rs35794377 | rs7736825 | rs17885353 |
| rs17878685 | rs12186362 | rs34935768 | rs16877442 | rs12656234 | rs17880343 | rs12188015 |
| rs2438608 | rs36052290 | rs16877466 | rs2913544 | rs9293797 | rs9293799 | rs17883985 |
| rs2918422 | rs2438607 | rs428279 | rs10514174 | rs2434300 | rs2170 | rs34579776 |
| rs17880038 | rs11273406 | rs2438606 | rs34102379 | rs17879824 | rs17882767 | rs10071934 |
| rs380747 | rs11951056 | rs17885253 | rs2434312 | rs391521 | rs17880126 | rs17882279 |
| rs2434313 | rs2434278 | rs17885404 | rs7736549 | rs2434314 | rs17878367 | rs2438647 |
| rs17879970 | rs9293798 | rs17885943 | rs13180294 | rs2438646 | rs2434315 | rs10590424 |
| rs10042207 | rs17879460 | rs12651918 | rs17885391 | rs1866389 | rs2438645 | rs13154820 |
| rs17883865 | rs10600128 | rs2434299 | rs10057390 | rs17879984 | rs17882932 | rs17882650 |
| rs2438605 | rs17879700 | rs4145069 | rs17885103 | rs2438604 | rs2434277 | rs34135437 |
| rs17883110 | rs34212380 | rs2438649 | rs365384 | rs17882871 | rs13355999 | rs689879 |
| rs17879000 | rs17878929 | rs11386965 | rs10447179 | rs443095 | rs2434298 | rs35079851 |
| rs10447180 | rs16877468 | rs10462572 | | | | |

ZNF627

| | | | | | | | |
|---|---|---|---|---|---|---|---|
| rs7253363 | rs35511396 | rs12975880 | rs4366815 | rs10408679 | rs35963942 | rs12972855 | rs28823955 |
| rs10406098 | rs35526749 | rs4804605 | rs11667775 | rs1673146 | rs35484790 | rs11665952 | rs34733225 |
| rs10415678 | rs34094922 | rs12462302 | rs28715023 | rs6511737 | rs35934908 | rs12979369 | rs34915809 |
| rs34763980 | rs34944783 | rs8110958 | rs12976530 | rs28446253 | rs10403331 | rs10425533 | rs35721267 |
| rs1471110 | rs12373534 | rs10403822 | rs5827129 | rs12460581 | rs1471111 | rs35697610 | rs7250667 |
| rs3035420 | rs4052626 | rs4804608 | rs35362984 | rs10408103 | rs4994983 | rs4052627 | rs4804609 |
| rs8100514 | rs10410181 | rs34274433 | rs12981552 | rs12985274 | rs36049863 | rs35214884 | rs2229532 |
| rs10409242 | rs8103510 | rs11879017 | rs36071847 | rs2229531 | rs35149487 | rs12972974 | rs34195347 |
| rs10418517 | rs2305799 | rs35113043 | rs10408325 | rs8106273 | rs12976766 | rs2328915 | rs10403399 |
| rs2607428 | rs12985407 | rs10418614 | rs2229530 | rs12972904 | rs35875992 | rs8105182 | rs34456522 |
| rs34375794 | rs35971218 | rs12981052 | rs8108668 | rs10426047 | rs35621512 | rs7246442 | rs10409095 |
| rs10417868 | rs10426263 | rs2071485 | rs10402720 | rs35838244 | rs10419625 | rs28373248 | rs2071484 |
| rs10404572 | rs10418463 | rs4239549 | rs10407232 | rs8107187 | rs8112083 | rs34316773 | rs35955762 |
| rs34437078 | rs2071483 | rs1263690 | rs35148340 | rs4804616 | rs28697222 | rs17001464 | rs10424332 |
| rs35877992 | rs4804617 | rs17001485 | rs3760780 | rs12980525 | rs12151212 | rs4052625 | rs10420734 |
| rs7256770 | rs12984577 | rs7256117 | rs7253275 | rs17001489 | rs7247136 | rs11551815 | rs1534561 |
| rs36034800 | rs10420009 | rs12973816 | rs9807866 | rs5827132 | rs10420316 | rs17001493 | rs12974843 |
| rs8105641 | rs1969533 | rs8111694 | rs12459055 | rs35879291 | rs8106114 | rs3923752 | rs8111700 |
| rs17001494 | rs36046884 | rs8105752 | rs4804171 | rs35825396 | rs8100206 | rs28544506 | rs8104902 |
| rs4804610 | rs34942751 | rs34289691 | rs34247688 | rs17001471 | rs28671573 | rs35379542 | rs12986317 |
| rs35572773 | rs8105144 | rs12978849 | rs7250798 | rs12980599 | rs10423235 | rs8106059 | rs12976980 |
| rs35031403 | rs12980663 | rs9305023 | rs8106186 | rs12978868 | rs28802306 | rs12971765 | rs11085785 |
| rs8106764 | rs12976994 | rs4052624 | rs12973498 | rs8108397 | rs9807915 | rs12986290 | rs10424122 |
| rs12980896 | rs11880512 | rs34718317 | rs12977012 | rs10416680 | rs12972003 | rs6511738 | rs12151062 |
| rs12978888 | rs36029549 | rs12974643 | rs12609030 | rs9807882 | rs12980021 | rs10418856 | rs8105395 |
| rs4804606 | rs9973204 | rs3922610 | rs8109499 | rs8111591 | rs11881292 | rs5827131 | rs4411616 |
| rs28641200 | rs35800992 | rs34228394 | rs34112728 | rs12162234 | rs28460406 | rs4804622 | rs35368398 |
| rs3035447 | rs4545929 | rs7256987 | rs7255169 | rs34225603 | rs9789280 | rs34589745 | rs34365612 |
| rs28452672 | rs35067254 | rs17448895 | rs34024878 | rs10407624 | rs28485477 | rs11085786 | rs7253448 |
| rs34419862 | rs10414382 | rs35624247 | rs11880143 | rs7249776 | rs4804611 | rs35685224 | rs4804623 |
| rs6511739 | rs7249892 | rs4804612 | rs11085788 | rs12983092 | rs889366 | rs11670781 | rs1128133 |
| rs34746623 | rs889367 | rs10415195 | rs7531 | rs11670877 | rs7508333 | rs11671741 | rs8105162 |
| rs34621855 | rs897811 | rs11668925 | rs8104957 | rs8108002 | rs8111258 | rs9973303 | rs8104211 |
| rs12973387 | rs35414678 | rs34132887 | rs4804613 | rs35954576 | rs4804607 | rs9973210 | rs4804614 |
| rs35779121 | rs10418695 | rs34711778 | rs4804615 | rs34328598 | rs7255562 | rs34843805 | rs34924329 |
| rs11666185 | rs10425114 | rs35357309 | rs7256301 | rs1263740 | rs12977542 | rs35675058 | rs34447952 |
| rs35349248 | rs12984228 | rs34110665 | rs35420552 | rs35909449 | rs12977773 | rs35448737 | rs2178224 |
| rs3865483 | rs34459704 | rs12461627 | rs2141399 | rs4804618 | rs35541942 | rs35315480 | rs2141400 |
| rs4804619 | rs34357745 | rs11672307 | rs35085568 | rs11882633 | rs35793693 | rs35864321 | rs10412992 |
| rs11882648 | rs3035423 | rs1263689 | rs8102091 | rs4804620 | rs2328916 | rs11085787 | rs8106713 |
| rs4804621 | rs4308060 | rs12976914 | rs8103576 | rs11878610 | rs35746002 | rs12978186 | rs2141398 |
| rs12459545 | | | | | | | |

IL1F10

| | | | | | | | |
|---|---|---|---|---|---|---|---|
| rs1138658 | rs4989178 | rs1665186 | rs1665193 | rs3213448 | rs4252017 | rs1688078 | rs3811050 |
| rs5833482 | rs13424580 | rs1627641 | rs1794065 | rs454078 | rs2121332 | rs3811051 | rs1867829 |
| rs2121329 | rs435381 | rs4251990 | rs380092 | rs6750555 | rs28928293 | rs1867830 | rs1665187 |
| rs417440 | rs4251991 | rs4252018 | rs6708096 | rs3811052 | rs34700180 | rs1665188 | rs315930 |
| rs4251992 | rs431726 | rs2264390 | rs4849149 | rs4848314 | rs13425255 | rs1630153 | rs416778 |
| rs452204 | rs2264097 | rs12469822 | rs17611872 | rs1665189 | rs315931 | rs416779 | rs3087266 |
| rs2264098 | rs4849150 | rs17042815 | rs9973741 | rs35204603 | rs11575824 | rs4252019 | rs2637991 |
| rs4849151 | rs13030546 | rs36121494 | rs315932 | rs2853628 | rs973635 | rs4848315 | rs4145013 |
| rs17042819 | rs2637993 | rs315933 | rs7559671 | rs315955 | rs12052825 | rs34510844 | rs6743171 |
| rs7579271 | rs17042917 | rs7587158 | rs440286 | rs12052833 | rs3811053 | rs13416494 | rs6746979 |
| rs10188601 | rs4251993 | rs4252040 | rs11123167 | rs3811054 | rs10188292 | rs2087705 | rs34263680 |
| rs7587166 | rs3087267 | rs1586815 | rs28928294 | rs11899198 | rs7596350 | rs374710 | rs7559883 |
| rs579543 | rs6721033 | rs3811055 | rs10176274 | rs13432148 | rs371590 | rs7587279 | rs315954 |
| rs35381256 | rs3811056 | rs17042827 | rs13410552 | rs17486819 | rs4251994 | rs4252020 | rs12471689 |
| rs4145014 | rs10199363 | rs1688077 | rs34235780 | rs11436108 | rs315953 | rs34032630 | rs3811057 |
| rs17042828 | rs4575729 | rs10712923 | rs35849018 | rs4252021 | rs2130991 | rs3827763 | rs6734238 |
| rs1618084 | rs315921 | rs7603907 | rs4252022 | rs2172189 | rs10669247 | rs34380841 | rs1618889 |
| rs373403 | rs315936 | rs315952 | rs11893774 | rs35217873 | rs6722922 | rs7562819 | rs34635610 |
| rs4251995 | rs4252023 | rs11684375 | rs3841013 | rs6750559 | rs34146986 | rs6723639 | rs4251996 |
| rs2232355 | rs6735388 | rs7608836 | rs11687782 | rs13026346 | rs373202 | rs4251997 | rs4252024 |
| rs12618462 | rs7569496 | rs35974997 | rs12711754 | rs383573 | rs315935 | rs4252025 | rs6736323 |
| rs28928295 | rs17042833 | rs12711755 | rs315920 | rs11575826 | rs4252026 | rs6721720 | rs3811058 |
| rs11684719 | rs13032281 | rs406124 | rs34932392 | rs315951 | rs6542117 | rs13406688 | rs35073604 |
| rs6715841 | rs384685 | rs4251998 | rs4252041 | rs11413284 | rs28928296 | rs34710796 | rs1688075 |
| rs4251954 | rs11306846 | rs4252027 | rs10635561 | rs6761821 | rs13398728 | rs1688076 | rs4251955 |
| rs1894405 | rs4252028 | rs35358603 | rs28928297 | rs13410964 | rs34195719 | rs4251956 | rs4251999 |
| rs4252029 | rs10661220 | rs6761276 | rs17042838 | rs34720511 | rs4251957 | rs4252000 | rs9005 |
| rs6542118 | rs6743376 | rs17042842 | rs34849245 | rs4251958 | rs11575827 | rs4252030 | rs6542119 |
| rs34320972 | rs4358126 | rs34832089 | rs4251959 | rs379155 | rs2592344 | rs6542120 | rs28928298 |
| rs13021292 | rs6542113 | rs4251960 | rs17042939 | rs4252031 | rs931471 | rs28928299 | rs7578112 |
| rs13387039 | rs4251961 | rs4252001 | rs3087268 | rs923692 | rs13005572 | rs7561598 | rs418217 |
| rs4252037 | rs315934 | rs396201 | rs2011678 | rs28928300 | rs7575402 | rs7573950 | rs4251962 |
| rs35225065 | rs315950 | rs902693 | rs28928301 | rs11891198 | rs7574159 | rs4251963 | rs392503 |
| rs4252032 | rs34177803 | rs28929168 | rs11886743 | rs35998927 | rs4251964 | rs3087262 | rs4252033 |
| rs6739871 | rs28928302 | rs11893386 | rs13390378 | rs4251965 | rs7607910 | rs4252034 | rs6739883 |
| rs28928303 | rs11886754 | rs7574427 | rs4251966 | rs7595789 | rs3087269 | rs3215028 | rs28928304 |
| rs6741180 | rs1794071 | rs4251967 | rs439154 | rs397211 | rs12475781 | rs28928305 | rs4496335 |
| rs13390577 | rs11677397 | rs7582194 | rs386745 | rs494089 | rs28928306 | rs6731551 | rs10207930 |
| rs4251968 | rs7598672 | rs4252042 | rs11690459 | rs6728590 | rs34670885 | rs17042923 | rs13422725 |
| rs4252035 | rs13011842 | rs13027999 | rs13432105 | rs2234676 | rs7598872 | rs315949 | rs6708535 |
| rs11684277 | rs13394316 | rs2234677 | rs7608130 | rs1388428 | rs11123159 | rs11683132 | rs13406085 |
| rs2234678 | rs7596007 | rs4252036 | rs28928307 | rs11677407 | rs1623119 | rs2234679 | rs3181051 |
| rs315948 | rs12468224 | rs11684289 | rs34483192 | rs16065 | rs4252002 | rs1388429 | rs34337721 |
| rs4368340 | rs17042888 | rs4251969 | rs7582732 | rs3087270 | rs35107184 | rs11688270 | rs1794069 |
| rs4251970 | rs2232352 | rs35803828 | rs28928308 | rs11684371 | rs34181521 | rs4252038 | rs4252003 |
| rs315947 | rs28928309 | rs35430960 | rs637936 | rs4251971 | rs2232353 | rs315946 | rs13386602 |
| rs11898742 | rs693498 | rs4252039 | rs4252004 | rs315945 | rs13398125 | rs5833483 | rs315922 |
| rs4251972 | rs2853629 | rs315944 | rs13389457 | rs35818660 | rs6542114 | rs4251973 | rs4252005 |
| rs3181059 | rs5833480 | rs11123161 | rs2592349 | rs4251974 | rs4252006 | rs315943 | rs28538191 |
| rs34717619 | rs440321 | rs4251975 | rs426476 | rs315942 | rs5833481 | rs12328766 | rs2592348 |
| rs4251976 | rs4252007 | rs3087271 | rs28628393 | rs2121326 | rs3978691 | rs315919 | rs3087263 |
| rs315941 | rs28711729 | rs12329129 | rs2855822 | rs4251977 | rs444413 | rs315940 | rs13424596 |
| rs12328368 | rs13382561 | rs4251978 | rs4252008 | rs315939 | rs13424676 | rs11681884 | rs2029582 |
| rs4251979 | rs4252009 | rs2902452 | rs13389666 | rs17669228 | rs17207494 | rs4251980 | rs34229798 |
| rs315938 | rs11886660 | rs28730394 | rs17042894 | rs4251981 | rs3181052 | rs6754298 | rs13424701 |
| rs28436104 | rs11473501 | rs4251982 | rs3181053 | rs315937 | rs13389803 | rs17042853 | rs34643047 |
| rs2637988 | rs35693848 | rs3099477 | rs11887823 | rs4849152 | rs315923 | rs2592347 | rs1794066 |
| rs2921717 | rs11891557 | rs7579943 | rs315924 | rs2254511 | rs1794067 | rs13417336 | rs12711750 |
| rs7596311 | rs7561080 | rs2855821 | rs4252010 | rs11123164 | rs35566948 | rs4849153 | rs28672736 |
| rs4251983 | rs1794068 | rs3099478 | rs11677043 | rs7596414 | rs34258774 | rs2592346 | rs1665190 |
| rs6759205 | rs11682107 | rs33997117 | rs33981313 | rs4251984 | rs419598 | rs36078521 | rs34920778 |
| rs10686567 | rs452699 | rs4251985 | rs423904 | rs3099479 | rs11693750 | rs6730516 | rs315925 |
| rs928940 | rs4252011 | rs2248588 | rs11677088 | rs7606121 | rs28648961 | rs4251986 | rs2637989 |
| rs35376823 | rs11678375 | rs7606142 | rs11677140 | rs4251987 | rs446433 | rs1374281 | rs12477866 |
| rs10185781 | rs10171849 | rs878972 | rs495282 | rs2248596 | rs12477867 | rs17042869 | rs1621602 |
| rs35564162 | rs2232354 | rs2248600 | rs12466799 | rs1542176 | rs1621603 | rs4251988 | rs495410 |
| rs2248604 | rs35405134 | rs12475887 | rs315926 | rs4251989 | rs34338955 | rs895496 | rs11123160 |
| rs11123162 | rs2637995 | rs28588003 | rs4252012 | rs17042998 | rs6759676 | rs7587033 | rs315927 |
| rs3053140 | rs4252013 | rs895495 | rs11382400 | rs13385228 | rs13404928 | rs13011389 | rs4252014 |
| rs11885498 | rs34124861 | rs11686467 | rs11695303 | rs11683422 | rs442710 | rs34192436 | rs35831508 |
| rs7559656 | rs7580634 | rs5833484 | rs2592345 | rs11887879 | rs35997925 | rs11686511 | rs35727625 |
| rs5833485 | rs408392 | rs315958 | rs10186133 | rs36023710 | rs497506 | rs1665191 | rs2071459 |
| rs11891927 | rs34730661 | rs11693683 | rs602927 | rs33993410 | rs4252015 | rs11123165 | rs7574787 |
| rs6738239 | rs11695584 | rs3978692 | rs447713 | rs315957 | rs12711751 | rs11896207 | rs454377 |
| rs3053142 | rs4252016 | rs13393926 | rs12711752 | rs6738377 | rs388500 | rs1665192 | rs128964 |
| rs34132411 | rs13409360 | rs1446509 | rs11891094 | rs33995342 | rs3087264 | rs35786438 | rs13409371 |
| rs11897481 | rs17042905 | rs34928804 | rs598859 | rs11123166 | rs17042810 | rs1446510 | rs315928 |
| rs377086 | rs3087265 | rs34218248 | rs10181720 | rs13431314 | rs414556 | rs33984161 | rs448341 |
| rs315956 | rs10184259 | rs13389431 | rs399826 | rs34154951 | rs434792 | rs2264096 | rs10169599 |
| rs35081995 | rs1688072 | rs3978693 | rs451578 | rs2172190 | rs4989179 | rs2121327 | rs315929 |
| rs6758355 | rs432014 | rs35944107 | | | | | |

SERPIN2

| | | | | | | | |
|---|---|---|---|---|---|---|---|
| rs13397106 | rs11418943 | rs12694626 | rs13388692 | rs12479146 | rs6739311 | rs35279856 | rs13426097 |
| rs13432278 | rs6742903 | rs13011032 | rs4574111 | rs11695803 | rs6436451 | rs7602990 | rs10625362 |
| rs13406925 | rs2118409 | rs7563931 | rs34726435 | rs13432690 | rs6704670 | rs7566799 | rs34240186 |
| rs13432693 | rs6704671 | rs4574110 | rs4674846 | rs11884404 | rs5839035 | rs1866152 | rs4674847 |
| rs34219787 | rs3080092 | rs1438828 | rs4674848 | rs11884535 | rs34034262 | rs12694627 | rs4674849 |
| rs34900198 | rs3080093 | rs12616221 | rs2099602 | rs13384685 | rs11326293 | rs4674839 | rs13429547 |
| rs13410227 | rs2118408 | rs4674840 | rs2083121 | rs13384766 | rs35728279 | rs4674841 | rs2083120 |
| rs12472341 | rs3080094 | rs1371028 | rs6708287 | rs6715768 | rs6718422 | rs11693563 | rs12478391 |
| rs36034130 | rs10524883 | rs720634 | rs2099601 | rs3795879 | rs3948261 | rs1821937 | rs10188083 |
| rs6747096 | rs7605903 | rs7560159 | rs10177142 | rs11548971 | rs6436453 | rs7560470 | rs11684839 |
| rs11548974 | rs6436454 | rs7574526 | rs11673799 | rs10170379 | rs1530021 | rs7560399 | rs13013387 |
| rs10183138 | rs1530020 | rs36006250 | rs10188383 | rs10196778 | rs1530019 | rs6436459 | rs11679799 |
| rs13004514 | rs35047534 | rs35647278 | rs13382587 | rs35905987 | rs7583799 | rs4674842 | rs7563863 |
| rs13392268 | rs7584131 | rs4674843 | rs7566629 | rs13392374 | rs7584132 | rs2083122 | rs7566640 |
| rs13392495 | rs7584056 | rs4674845 | rs7580846 | rs13392412 | rs6436455 | rs12619651 | rs7580849 |
| rs13392722 | rs13007502 | rs34770432 | rs11675207 | rs1866153 | rs6436456 | rs36076279 | rs11675323 |
| rs6726753 | rs6436457 | rs35865010 | rs11681120 | rs10177151 | rs7587909 | rs7601097 | rs4674850 |
| rs10189547 | rs7588220 | rs35387795 | rs4674851 | rs6742620 | rs4674837 | rs11695741 | rs4674852 |
| rs10203588 | rs6436458 | rs34478453 | rs11381825 | rs3795877 | rs7590948 | rs11678628 | rs11283961 |
| rs3839039 | rs35805149 | rs13393673 | rs11336057 | rs7581619 | rs6742225 | rs10933032 | rs34235567 |
| rs34078713 | rs11403950 | rs13015494 | rs13411332 | rs11548973 | rs11692680 | rs2037755 | rs4368321 |
| rs3795875 | rs13412535 | rs35298522 | rs12436 | rs4674838 | rs2037754 | rs11902594 | rs16865466 |
| rs1438829 | rs10706128 | rs17196253 | rs1438830 | rs10545713 | rs7602039 | rs34907719 | rs10206895 |
| rs7576030 | rs4583455 | rs10183743 | rs7602765 | rs13022548 | rs10184062 | rs7602769 | rs4583456 |

LGALS2

| | | | | | | | |
|---|---|---|---|---|---|---|---|
| rs2076087 | rs35513222 | rs5756737 | rs9610805 | rs2281099 | rs5756738 | rs2076088 | rs34247666 |
| rs5756739 | rs2281096 | rs12484334 | rs140058 | rs8138741 | rs8141621 | rs35459261 | rs8139004 |
| rs2235338 | rs5756740 | rs12158451 | rs2281097 | rs4821669 | rs12158219 | rs2235339 | rs4821670 |
| rs9607472 | rs28528864 | rs4821671 | rs11424589 | rs6000802 | rs4821672 | rs34771139 | rs2281100 |
| rs4821673 | rs8138795 | rs9607475 | rs6000804 | rs8138909 | rs35991299 | rs6000805 | rs35544110 |
| rs34552358 | rs6000806 | rs9610806 | rs9607476 | rs35861132 | rs5756729 | rs35711326 | rs34746744 |
| rs12158527 | rs34520656 | rs6000808 | rs5756730 | rs34614491 | rs12159678 | rs10588992 | rs12484152 |
| rs11089845 | rs5756731 | rs10633102 | rs5750451 | rs5756732 | rs33964668 | rs7290697 | rs5756733 |
| rs11912616 | rs5750452 | rs10427826 | rs11913057 | rs5750453 | rs11542010 | rs5750450 | rs5750454 |
| rs2281098 | rs34734674 | rs7291162 | rs10427607 | rs35129481 | rs10574720 | rs13055845 | rs140057 |
| rs10616872 | rs13056859 | rs11311539 | rs10617077 | rs13055511 | rs6000803 | rs7291467 | rs13057024 |
| rs5756736 | | | | | | | |

### INDUSTRIAL APPLICATION

The present invention is directed to methods for assessing a subject's risk of developing ACS. The methods comprise the analysis of polymorphisms herein shown to be associated with increased or decreased risk of developing ACS, or the analysis of results obtained from such an analysis. The use of polymorphisms herein shown to be associated with increased or decreased risk of developing ACS in the assessment of a subject's risk are also provided, as are nucleotide probes and primers, kits, and microarrays suitable for such assessment. Methods of treating subjects having the polymorphisms herein described are also provided. Methods for screening for compounds able to modulate the expression of genes associated with the polymorphisms herein described are also provided.

All patents, publications, scientific articles, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods described herein are representative of various embodiments or preferred embodiments and are exemplary only and not intended as limitations on the scope of the invention. Other objects, aspects, examples and embodiments will occur to those skilled in the art upon consideration of this specification, and are encompassed within the spirit of the invention as defined by the scope of the claims. It will be readily apparent to one skilled in the art that varying substitutions and modifications can be made to the invention disclosed herein without departing from the scope and spirit of the invention. The invention illustratively described herein suitably can be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification, thus indicating additional examples, having different scope, of various alternative embodiments of the invention. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention.

1-69. (canceled)

70. A method of determining a subject's risk of developing ACS, comprising analyzing a sample from said subject for the presence or absence of at least one polymorphism selected from the group consisting of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2);
372 T/C in the gene encoding Tissue inhibitor of metalloproteinase 1 (TIMP1);
and
a polymorphism in linkage disequilibrium with any one of said polymorphisms,
wherein the presence or absence of said at least one polymorphism is indicative of the subject's risk of developing ACS.

71. The method of claim 70, wherein the method further comprises analyzing said sample for the presence or absence of at least one polymorphism selected from the group consisting of:
−509 C/T in the gene encoding Transforming growth factor β1 (TGFB1);
Thr26Asn A/C in the gene encoding Lymphotoxin α (LTA);
Asp299Gly A/G in the gene encoding Toll-like Receptor 4 (TLR4);
Thr399Ile C/T in the gene encoding TLR4;
−63 T/A in the gene encoding Nuclear factor of kappa light polypeptide gene enhancer in B-cells inhibitor-like 1 (NFKBIL1);
−1630 Ins/Del (AACTT/Del) in the gene encoding Platelet derived growth factor receptor alpha (PDGFRA);
−1607 1G/2G (Del/G) in the gene encoding Matrix metalloproteinase 1 (MMP1);
12 IN 5 C/T in the gene encoding Platelet derived growth factor alpha (PDGFA);
−588 C/T in the gene encoding Glutamate-cysteine ligase modifier subunit (GCLM);
Ile132Val A/G in the gene encoding Olfactory receptor analogue OR13G1 (OR13G1);
Glu288Val A/T (M/S) in the gene encoding alpha 1-antitrypsin (α1-AT);
K(469E A/G in the gene encoding Intracellular adhesion molecule 1 (ICAM1);
−23 C/G in the gene encoding HLA-B associated transcript 1 (BAT1);
Glu298Asp C/T in the gene encoding Nitric Oxide synthase 3 (NOS3);
−668 4G/5G in the gene encoding Plasminogen activator inhibitor 1 (PAI-1);
−181 A/G in the gene encoding Matrix metalloproteinase 7 (MMP7); and
a polymorphism in linkage disequilibrium with any one of said polymorphisms.

72. The method of claims 70 or 71, wherein said method comprises the analysis of one or more epidemiological risk factors.

73. A method of determining a subject's risk of developing ACS, said method comprising the steps of:
(i) obtaining the result of one or more genetic tests of a sample from said subject; and
(ii) analyzing the result for the presence or absence of at least one polymorphism selected from the group consisting of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2); or
372 T/C in the gene encoding Tissue inhibitor of metalloproteinase 1 (TIMP1); and
a polymorphism in linkage disequilibrium with any one of said polymorphisms,
wherein a result indicating the presence or absence of said at least one polymorphism is indicative of the subject's risk of developing ACS.

74. The method of claim 73, wherein a result indicating the presence of at least one polymorphism selected from the group consisting of:
the Ser52Ser (223 C/T) CC genotype in the gene encoding FGF2;
the Q576R A/G AA genotype in the gene encoding IL4RA;
the Hom T2437C CC or CT genotype in the gene encoding HSP70;
the 874 A/T TT genotype in the gene encoding IFNG;
the −589 C/T CT or TT genotype in the gene encoding IL-4;
the −1084 A/G GG genotype in the gene encoding IL-10;
the Arg213Gly C/G CG or GG genotype in the gene encoding SOD3;
the Asn 125 Ser AG or GG genotype in the gene encoding Cathepsin G; and
372 T/C TT genotype in the gene encoding TIMP1
is indicative of a reduced risk of developing ACS.

75. The method of claim 73, wherein a result indicating the presence of at least one polymorphism selected from the group consisting of:
the −1903 A/G GG genotype in the gene encoding CMA1;
the −82 A/G GG genotype in the gene encoding MMP12;
the +459 C/T Intron 1 CT or TT genotype in the gene encoding MIP1A;
the Asn 125 Ser AA genotype in the gene encoding Cathepsin G;
the I249V TT genotype in the gene encoding CX3CR1;
the Gly 881 Arg G/C CC or CG genotype in the gene encoding NOD2; and
the 372 T/C CC genotype in the gene encoding TIMP1
is indicative of an increased risk of developing ACS.

76. A nucleotide probe and/or primer, wherein the nucleotide probe and/or primer spans, or is capable of spanning, a polymorphic region of a gene comprising a polymorphism selected from the group of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2);
372 T/C in the gene encoding Tissue inhibitor of metalloproteinase 1 (TIMP1);
−509 C/T in the gene encoding Transforming growth factor β1 (TGFB1);
Thr26Asn A/C in the gene encoding Lymphotoxin α (LTA);
Asp299Gly A/G in the gene encoding Toll-like Receptor 4 (TLR4);
Thr399Ile C/T in the gene encoding TLR4;
−63 T/A in the gene encoding Nuclear factor of kappa light polypeptide gene enhancer in B-cells inhibitor-like 1 (NFKBIL1);
−1630 Ins/Del (AACTT/Del) in the gene encoding Platelet derived growth factor receptor alpha (PDGFRA);
−1607 1G/2G (Del/G) in the gene encoding Matrix metalloproteinase 1 (MMP1);
12 IN 5 C/T in the gene encoding Platelet derived growth factor alpha (PDGFA);
−588 C/T in the gene encoding Glutamate-cysteine ligase modifier subunit (GCLM);
Ile132Val A/G in the gene encoding Olfactory receptor analogue OR13G1 (OR13G1);
Glu288Val A/T (M/S) in the gene encoding alpha 1-antitrypsin (α1-AT);
K469E A/G in the gene encoding Intracellular adhesion molecule 1 (ICAM1);
−23 C/G in the gene encoding HLA-B3 associated transcript 1 (BAT1);
Glu298Asp G/T in the gene encoding Nitric Oxide synthase 3 (NOS3);
−668 4G/5G in the gene encoding Plasminogen activator inhibitor 1 (PAI-1);
−181 A/G in the gene encoding Matrix metalloproteinase 7 (MMP7); and
a polymorphism in linkage disequilibrium with any one of said polymorphisms.

77. The nucleotide probe and/or primer of claim 76, comprising a sequence selected from the group of: SEQ. ID. NOs.1-124.

78. A nucleic acid microarray, comprising a substrate that presents nucleic acid sequences capable of hybridizing to nucleic acid sequences which encode at least one polymorphism selected from the group selected from:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2);
372 T/C in the gene encoding Tissue inhibitor of metalloproteinase 1 (TIMP1); and
a polymorphism in linkage disequilibrium with any one of said polymorphisms or a sequence complimentary thereto.

79. An antibody microarray, comprising a substrate that presents antibodies capable of binding to a gene expression product that is upregulated or downregulated when associated with a polymorphism selected from the group of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2);
372 T/C in the gene encoding Tissue inhibitor of metalloproteinase 1 (TIMP1);
−509 C/T in the gene encoding Transforming growth factor β1 (TGFB 1);
Thr26Asn A/C in the gene encoding Lymphotoxin α (LTA);
Asp299Gly A/G in the gene encoding Toll-like Receptor 4 (TLR4);
Thr399Ile C/T in the gene encoding TLR4;
−63 T/A in the gene encoding Nuclear factor of kappa light polypeptide gene enhancer in B-cells inhibitor-like 1 (NFKBIL1);
−1630 Ins/Del (AACTT/Del) in the gene encoding Platelet derived growth factor receptor alpha (PDGFRA);
−1607 1G/2G (Del/G) in the gene encoding Matrix metalloproteinase 1 (MMP1);
12 IN 5 C/T in the gene encoding Platelet derived growth factor alpha (PDGFA);
−588 C/T in the gene encoding Glutamate-cysteine ligase modifier subunit (GCLM);
Ile132Val A/G in the gene encoding Olfactory receptor analogue OR13G1 (OR13G1);
Glu288Val A/T (M/S) in the gene encoding alpha 1-antitrypsin (α1-AT);
K469E A/G in the gene encoding Intracellular adhesion molecule 1 (ICAM1);
−23 C/G in the gene encoding HLA-B associated transcript 1 (BAT1);
Glu298Asp G/T in the gene encoding Nitric Oxide synthase 3 (NOS3);
−668 4G/5G in the gene encoding Plasminogen activator inhibitor 1 (PAI-1);
−181 A/G in the gene encoding Matrix metalloproteinase 7 (MMP7); and
a polymorphism in linkage disequilibrium with any one of said polymorphisms.

80. A method for screening for compounds that modulate the expression and/or activity of a gene, the expression of which is upregulated or downregulated when associated with a protective polymorphism selected from the group defined in claim 74 or a susceptibility polymorphism selected from the group defined in claim 75, said method comprising the steps of:
contacting a candidate compound with a cell comprising a susceptibility or protective polymorphism associated with the upregulation or downregulation of expression of a gene; and
measuring the expression of said gene following contact with said candidate compound,
wherein a change in the level of expression after the contacting step as compared to before the contacting step is indicative of the ability of the compound to modulate the expression and/or activity of said gene.

81. The method of claim 80, wherein said cell is a human vascular cell which has been pre-screened to confirm the presence of said polymorphism, or which has been pre-screened to confirm the presence, and baseline level of expression, of said gene.

82. The method of claim 80 or 81, wherein said cell comprises a susceptibility polymorphism associated with upregulation of expression of said gene and said screening is for candidate compounds which downregulate expression of said gene.

83. The method of claim 80 or 81, wherein said cell comprises a susceptibility polymorphism associated with downregulation of expression of said gene and said screening is for candidate compounds which upregulate expression of said gene.

84. The method of claim 80 or 81, wherein said cell comprises a protective polymorphism associated with upregulation of expression of said gene and said screening is for candidate compounds which further upregulate expression of said gene.

85. The method of claim 80 or 81, wherein said cell comprises a protective polymorphism associated with downregulation of expression of said gene and said screening is for candidate compounds which further downregulate expression of said gene.

86. A method of assessing the likely responsiveness of a subject predisposed to or diagnosed with ASC to a prophylactic or therapeutic treatment, which treatment involves restoring the physiologically active concentration of a product of gene expression to be within a range which is normal for the age and sex of the subject, the method comprising detecting in said subject the presence or absence of a susceptibility polymorphism selected from the group defined in claim 75 which when present either upregulates or downregulates expression of said gene such that the physiological active concentration of the expressed gene product is outside said normal range, wherein the detection of the presence of said polymorphism is indicative of the subject likely responding to said treatment.

87. A kit for assessing a subject's risk of developing ACS, said kit comprising a means of analyzing a sample from said subject for the presence or absence of at least one polymorphism selected from the group consisting of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2); or
372 T/C in the gene encoding Tissue inhibitor of metalloproteinase 1 (TIMP1); and
a polymorphism in linkage disequilibrium with any one of said polymorphisms.
